(19) United States
(12) Patent Application Publication
(10) Pub. No.: US 2024/0116994 A1
(43) Pub. Date: Apr. 11, 2024

(54) Peptides And Use Thereof For Diagnosing And Treating Antiphospholipid Syndrome

(71) Applicants: UNIVERSITE DE GENEVE, Geneve (CH); HOPITAUX UNIVERSITAIRES DE GENEVE, Geneve (CH)

(72) Inventors: Karim BRANDT, Geneve (CH); Francois MACH, Geneve (CH); Marc Casra MOGHBEL, Aubonne (CH)

(21) Appl. No.: 18/282,348

(22) PCT Filed: Mar. 17, 2022
(86) PCT No.: PCT/EP2022/057085

§ 371 Date: Sep. 15, 2023

(30) Foreign Application Priority Data
Mar. 18, 2021 (EP) 21163557.8

(51) Int. Cl. C07K 14/47; A61P 37/06; C07K 16/18; G01N 33/68

(52) U.S. Cl. CPC C07K 14/4713; A61P 37/06; C07K 16/18; G01N 33/6893; A61K 38/00

(57) ABSTRACT

The invention provides an isolated peptide and a peptide construct of formula I that binds antiphospholipid antibodies (aPLA). The present invention further provides methods for detection of aPLA. The present invention also provides methods for diagnosing and treating the antiphospholipid syndrome (APS).

### FIELD OF THE INVENTION

The invention provides a peptide construct of formula I that binds antiphospholipid antibodies (aPLA). The present invention further provides methods for detection of aPLA. The present invention also provides methods for diagnosing and treating the antiphospholipid syndrome (APS).

### BACKGROUND OF THE INVENTION

The antiphospholipid syndrome (APS) is described as a common risk factor for recurrent thromboembolic events and/or pregnancy complications resulting from circulating antiphospholipid antibodies (aPLA). It is now widely accepted that the plasma phospholipid binding protein β-2-Glycoprotein 1 ((β2GP1) is the main antigenic target for aPLA. β2GP1 is a protein of 43 kDa composed of 5 short consensus repeat domains called “sushi” domains. Two different conformations exist for β2GP1: a circular plasma conformation and a fishhook conformation. Epitopes within domains I and V are involved in maintaining a circular conformation, whereas binding of domain V to anionic surfaces induces a fishhook conformation and exposure of a cryptic epitope in domain I. This cryptic epitope is described as being located around residues 39 and 43; however, Iverson et al. have identified additional residues involved in the recognition by pathogenic anti-β2GP1 antibodies in domain I. Ioannou et al. have also studied mutations including residues R39 to R43 describing complex and probably discontinuous epitopes. Their data suggest that the epitope(s) are not “classical” or that several epitopes are present in domain I and could potentially even be present elsewhere in β2GP1.

Humoral immunophysiology studies of APS and the treatment of APS patients with an anti-CD20 monoclonal antibody (rituximab) have aroused interest in B cells as therapeutic targets. Anti-CD20-treated APS patients have a normal distribution of anti-β2GP1, anticardiolipin (aCL) and Lupus anticoagulant (LAC) antibody titers and improved clinical manifestations. The isotype of anti-β2GP1 antibody is mainly IgG, suggesting that the production of these antibodies requires antigen-specific CD4⁺ T helper cells.

In addition, even though different tools are used, the diagnosis of APS patients currently is laborious and time-consuming. Indeed, the current diagnosis requires the presence of a thrombotic or obstetrical complication and an elevation of antibodies anticardiolipin or anti-β2-glycoprotein-1 at two different samples, spaced 12 weeks. Antibody levels are measured using ELISA assays that have numerous standardization and antigen conformation challenges (current ELISA assays use an entire segment of (β2-glycoprotein-1 protein) inducing false negative (<42% sensitivity). The exact target of anti-β2-glycoprotein-1 antibodies is not well identified, there was no way to standardize the quantification of antibodies.

Hence, there is a need for a diagnostic tool and method that allows for improved diagnosis of APS, in particular with high sensitivity and high specificity.

### SUMMARY OF THE INVENTION

Thus an aspect of the present invention provides an isolated peptide comprising the amino acid sequence X₁SRGGMRKX₂KKX₃X₄TX₅ (SEQ ID NO: 1), wherein
X₁ is R or V,
X₂ is R or K,
X₃ is P or K,
X₄ is L or K,
X₅ is G or K.

A further aspect of the present invention provides a peptide construct of formula I wherein

S₂—P—S₁—P (I)

P is a peptide consisting of the amino acid sequence X₁SRGGMRKX₂KKX₃X₄TX₅(SEQ ID NO: 1), wherein
X₁ is R or V,
X₂ is R or K,
X₃ is P or K,
X₄ is L or K,
X₅ is G or K.

S₂ is absent or is a spacer peptide sequence or a polymer, wherein the spacer peptide sequence is a poly-Gly spacer consisting of 3-16 glycines or a 3 to 16-amino acid Gly-rich spacer;

S₁ is a spacer peptide sequence or a polymer, wherein the spacer peptide sequence is a poly-Gly spacer consisting of 3-16 glycines or a 3 to 16-amino acid Gly-rich spacer.

Another aspect of the present invention provides a peptide of the invention or a peptide construct of formula I of the invention for use in a method for diagnosing of an antiphospholipid syndrome (APS) in a subject, wherein presence or absence of an antiphospholipid antibody (aPLA) is detected in a sample from the subject diagnosed, and wherein the presence of an antiphospholipid antibody is indicative of the APS disease and wherein the antiphospholipid antibody is detected using an immunoassay comprising the steps of
(i) providing a sample;
(ii) contacting the sample with the peptide of the invention immobilized on a surface or on beads or the peptide construct of formula I of the invention immobilized on a surface or on beads, under conditions allowing for the formation of a complex between antiphospholipid antibodies with the peptide of the invention or the peptide construct of formula I of the invention;
(iii) detecting the complex.

Another aspect of the present invention provides a method for detecting the presence of antiphospholipid antibody in a sample comprising
(i) providing a sample,
(i) contacting the sample with the peptide of the invention immobilized on a surface or on beads or the peptide construct of formula I of the invention immobilized on a surface or on beads, under conditions allowing for the formation of a complex between antiphospholipid antibodies with the peptide of the invention or the peptide construct of formula I of the invention;
(iii) detecting the complex using an immunoassay.

Another aspect of the present invention provides an antibody or an antigen-binding fragment thereof, that binds to the peptide of the invention or binds to the peptide construct of formula I of the invention.

Another aspect of the present invention provides a kit for detecting in a sample the presence or absence of an antiphospholipid antibody, the kit comprising the peptide of the invention or the peptide construct of formula I of the invention.

Another aspect of the present invention provides a device for detecting in a sample the presence or absence of an antiphospholipid antibody, the device comprising a solid support comprising the peptide of the invention or the peptide construct of formula I of the invention.

Another aspect of the present invention provides a pharmaceutical composition comprising the peptide of the invention or the peptide construct of formula I of the invention in an amount effective to prevent, reduce or inhibit one or more symptoms of the antiphospholipid syndrome (APS) in a subject in need thereof, and a pharmaceutically acceptable carrier for administration of the peptide or the peptide construct.

Another aspect of the present invention provides a peptide of the invention, a peptide construct of formula I of the invention or a pharmaceutical composition of the invention for use in a method for preventing and/or inhibiting one or more symptoms of the antiphospholipid syndrome (APS) in a subject.

Another aspect of the present invention provides a peptide of the invention or the peptide construct of formula I of the invention for use in a method of selectively removing antiphospholipid antibodies from blood, serum or plasma comprising the steps of immobilizing the peptide of the invention or the peptide construct of formula I of the invention to an immunoaffinity membrane and passing blood, serum or plasma through said immunoaffinity membrane so that antiphospholipid antibodies from the blood, serum or plasma will be removed by the immunoaffinity membrane.

### BRIEF DESCRIPTION OF THE FIGURES

FIG. 1 shows epitope recognized by aPL requires to be spatially oriented for optimal interactions—A) Functional evaluation with reduced β2GP1 ((β2GP1), Domain I-II of β2GP1 (Dom I-II), peptide R39-R43, peptide Ia-1 and Ib-1 of aPL at 1/10 dilution of aPL pool. Data are represented in mean of fold increased±s.e.m relative to interaction level with reduced β2GP1 ((β2GP1). (n=9) B) Function comparison between interactions of biotinylated- (Pept-Biot) and nude-peptide (Pept-Nude) with aPL at 1/10 and 1/1,000 dilution of aPL pool. (n=9) C) Quantification of aPL interactions at 1/100, 1/1,000 and 1/10,000 dilution with β2GP1 wild-type epitope (R39-43-biot) (Upper panel) and monomeric peptide Ib-1-biot (Bottom panel) by surface plasma resonance. Representative graph of 3 independent experiments D) Quantification of aPL interactions at 1/100, 1/1,000 and 1/10,000 dilution with dimeric peptide Ib-1-biot (Bottom panel) by surface plasma resonance. Representative graph of 3 independent experiments E) Function comparison between interactions of monomeric (Ib-1-biot) and dimeric peptide (Ib-1-biot-2x) and nude-peptide (Ib-1) (Pept-Nude) with aPL at 1/10 and 1/1,000 dilution of aPL pool. (n=9). The nonparametric Mann-Whitney U test was used for statistical analysis: *: p≤0.05; **: p≤0.005; ***: p≤0.0005. All data were represented as mean±s.e.m.

FIG. 2 shows surrounding part of epitope-determining sequence for aPL is essential for the proper interactions with epitope—A) Functional evaluation with identified peptides at indicated dilutions of aPL pool. Data are represented in mean of fold increased±s.e.m relative to interaction level with Ib-1-biot-2x (Ib-1.0-biot-2x). B) Upper panel, Dilution assay with selected peptides. Data are represented in mean of B/B0 (signal of aPL (pool)/signal of pool of plasma from healthy donors)±s.e.m relative to interaction level with reduced β2GP1 ((β2GP1). Doted-line=3×stdev of signal for 1/1,000 R39-R43. (n=10). Bottom panel, Quantification of AUC relative to responses of each peptides. C) Quantification of aPL interactions at 1/100, 1/1,000 and 1/10,000 dilution with peptide IIa-8.0-biot-2x (Upper panel) and monomeric peptide Ib-1-biot (Bottom panel) by surface plasma resonance. Quantification of AUC relative to responses of aPL to β2GP1 wild-type epitope (R39-43-biot), Ib-1-biot-2x and IIa-8.0-biot-2x (bottom panel). The nonparametric Mann-Whitney U test was used for statistical analysis: *: p≤0.05; **: p≤0.005; ***: p≤0.0005. All data were represented as mean±s.e.m.

FIG. 3 shows levels of circulating IgG anti-8.0-biot-2x in lupus-prone mouse model and Human cohort are strongly correlated with clinical manifestation of APS—Bar graphs represent the median±s.e.m. of (A) IgG anti-dsDNA, IgG anti-ApoA-1 and IgG anti-IIa-8.0-biot-2x autoantibody quantification in the serum, measured as optical density (OD) in Apoe^{-/-} or Apoe^{-/-}Nba2.Yaa mice on HCD (n=8-10 mice/group). All data were represented as mean±s.e.m. The nonparametric Mann-Whitney U test was used for statistical analysis: *: p≤0.05; ***: p≤0.0005. Spearman's rank correlation coefficients between IgGIIa-8.0-biot-2x and IgG anti-dsDNA or IgG anti-ApoA-1 (B), Platelets, RBC, kidney and mice weight (C), kidney and mice weight (D), and Fibrous cap thickness, total collagen and pro-MMP9 (E). HemosIL AcuStar anti-β2GP1 and IIa-8.0-biot-2x ROC curve comparison for Human cohort (F). Spearman's rank correlation coefficients between HemosIL AcuStar anti-β2GP1 and IIa-8.0-biot-2x (n=380) (G). All data were represented as mean±s.e.m. The nonparametric Mann-Whitney U test was used for statistical analysis: *: p≤0.05; **: p≤0.005; ***: p≤0.0005.

FIG. 4 shows the calibration curve using the antibody of the invention.

FIG. 5 shows optimized peptides are able to inhibit the binding activity of aPL in vitro—A) Quantification of reactivity against peptide IIa-8.0-biot-2x of aPL pool sera previously incubated with increasing concentration of monomeric IIa-5.0, IIa-7.1 and IIa-8.0 peptides. All data were represented as mean±s.e.m. (n=3 to 4) B) Quantification of reactivity against peptide IIa-8.0-biot-2x of aPL pool sera previously incubated with increasing concentration of dimeric IIa-5.0-2x, IIa-7.1-2x and IIa-8.0-2x peptides. (n=3) C) Quantification of reactivity against peptide IIa-8.0-biot-2x of aPL pool sera previously incubated with increasing concentration of dimeric IIa-5.0-2x, IIa-7.1-2x and IIa-8.0-2x-associated beads. All data were represented as mean±s.e.m. (n=3) The nonparametric Mann-Whitney U test was used for statistical analysis: *: p≤0.05; **: p≤0.005.

FIG. 6 shows graphical representation of peptide substitution scan microarray performed with four different aPL (Patient 1; 2; 3 and 4) and a pool of 11 patients (Pool) at 500 μg/ml. Five identified peptides with higher avidity for aPL. Representative data of fluorescence unit (A:U) relative to interaction level with R39-R43 (Upper Sequence).

FIG. 7 shows graphical representation of peptide substitution scan microarray performed with four different aPL (Patient 1; 2; 3 and 4) and a pool of 11 patients (Pool) at 500 μg/ml. Seven identified peptides with higher avidity for aPL. Data are represented in mean of fold increased±s.e.m relative to interaction level with Ib-1-biot-2x (Ib-1.0-biot-2x).

### DETAILED DESCRIPTION OF THE INVENTION

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise” is generally used in the sense of include, that is to say permitting the presence of one or more features or components. Also as used in the specification and claims, the language “comprising” can include analogous embodiments described in terms of “consisting of” and/or “consisting essentially of”.

As used in the specification and claims, the singular forms “a”, “an” and “the” include plural references unless the context clearly dictates otherwise.

As used in the specification and claims, the term “and/or” used in a phrase such as “A and/or B” herein is intended to include “A and B”, “A or B”, “A”, and “B”.

As used herein, an “amino acid”, “amino acid molecule” or “amino acid residue” refers to any naturally occurring amino acid, any amino acid derivative or any amino acid mimic known in the art, including modified or unusual amino acids. In certain embodiments, the residues of the peptide are sequential, without any non-amino acid interrupting the sequence of amino acid residues. In other embodiments, the sequence may comprise one or more non-amino acid moieties, for example spacers or linkers.

As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder, such as the antiphospholipid syndrome (APS). In other embodiments, the subject is a subject in need to prevent and/or inhibit symptoms of the antiphospholipid syndrome (APS). The term does not denote a particular age or sex. Thus, adult and newborn subjects, whether male or female, are intended to be covered.

An aspect of the invention provides an isolated peptide comprising the amino acid sequence X₁SRGGMRKX₂KKX₃X₄TX₅ (SEQ ID NO: 1), wherein
X₁ is R or V,
X₂ is R or K, preferably X₂ is K
X₃ is P or K, preferably X₃ is K
X₄ is L or K, preferably X₄ is K
X₅ is G or K, preferably X₅ is K

In some embodiments, the isolated peptide of the invention comprises the sequence selected from the group comprising:

(SEQ ID NO: 2)
RSRGGMRKRKKPLTG (IIa-8.0)
(SEQ ID NO: 3)
VSRGGMRKRKKPLTK (IIa-5.0)
(SEQ ID NO: 4)
VSRGGMRKRKKKKTG (IIa-7.1)
(SEQ ID NO: 5)
VSRGGMRKRKKPLTG (Ib-2.0)
(SEQ ID NO: 6)
VSRGGMRKKKKPLTG (Ib-1.0)
(SEQ ID NO: 7)
RSRGGMRKKKKPLTG (IIa-4.0)
(SEQ ID NO: 8)
VSRGGMRKKKKKKTG (IIa-3.1)
(SEQ ID NO: 9)
VSRGGMRKKKKPLTK (IIa-1.0)

In some embodiments, the isolated peptide of the invention comprises RSRGGMRKRKKPLTG (SEQ ID NO:2) (IIa-8.0).

In an embodiment of the invention, the isolated peptide consists of the amino acid sequence X₁SRGGMRKX₂KKX₃X₄TX₅ (SEQ ID NO: 1), wherein
X₁ is R or V,
X₂ is R or K, preferably X₂ is K
X₃ is P or K, preferably X₃ is K
X₄ is L or K, preferably X₄ is K
X₅ is G or K, preferably X₅ is K

In some embodiments, the isolated peptide of the invention consists of the sequence selected from the group comprising:

(SEQ ID NO: 2)
RSRGGMRKRKKPLTG (IIa-8.0)
(SEQ ID NO: 3)
VSRGGMRKRKKPLTK (IIa-5.0)
(SEQ ID NO: 4)
VSRGGMRKRKKKKTG (IIa-7.1)
(SEQ ID NO: 5)
VSRGGMRKRKKPLTG (Ib-2.0)
(SEQ ID NO: 6)
VSRGGMRKKKKPLTG (Ib-1.0)
(SEQ ID NO: 7)
RSRGGMRKKKKPLTG (IIa-4.0)
(SEQ ID NO: 8)
VSRGGMRKKKKKKTG (IIa-3.1)
(SEQ ID NO: 9)
VSRGGMRKKKKPLTK (IIa-1.0)

In some embodiments, the isolated peptide consists of RSRGGMRKRKKPLTG (SEQ ID NO:2) (IIa-8.0).

In some embodiments, the isolated peptide of the invention has the reversed amino acid sequence (reading from C- to N-terminus), namely X₅-T-X₄-X₃-K-K-X₂-KRMGGRS-X₁ (SEQ ID NO: 26).

In another embodiment, the invention provides a peptide construct consisting of the isolated peptide of the invention and a spacer at N- and/or C-terminus, wherein the spacer at N- and/or C-terminus is independently selected from a spacer peptide sequence having a length of at least 3 or at least 5 amino acid residues or a polymer.

In some embodiments the spacer peptide sequence comprises any amino acid residue, preferably any natural L-amino acid residue. In some other embodiments, the spacer peptide sequence is poly-Gly spacer, consisting of for example 3-16 or 5-16 glycines, or Gly-rich spacer, preferably 3 to 16-amino acids Gly-rich spacer or 5 to 16-amino acids Gly-rich spacer. In some embodiments, the spacer peptide sequence is a poly-Gly spacer consisting of 3-16 glycines or a 3 to 16-amino acids Gly-rich spacer;

In some embodiments, the spacer peptide sequence is selected from the group comprising GGGGSLVPRGSGGGGS (SEQ ID NO:10), GSGSGS (SEQ ID NO:11), GSGGGTGGGSG (SEQ ID NO:12), GGGGSGGGGS (SEQ ID NO:13), GGGGS (SEQ ID NO:14), GSGSGTGSGS (SEQ ID NO:15).

In other embodiments, the spacer is a polymer selected from the group comprising DSG, DSS, BS3, TSAT (trifunctional), BS(PEG)5, BS(PEG)9, DSP, DTSSP, DST, BSOCOES, EGS, Sulfo-EGS, DMA, DMP, DMS, DTBP, DFDNB, BMOE, BMB, BMH, TMEA (trifunctional), BM(PEG)2, BM(PEG)3, DTME, AMAS, BMPS, GMBS and Sulfo-GMBS, MBS and Sulfo-MBS, SMCC and Sulfo-SMCC, EMCS and Sulfo-EMCS, SMPB and Sulfo-SMPB, SMPH, LC-SMCC, Sulfo-KMUS, SM(PEG)2, SM(PEG)4, SM(PEG)6, SM(PEG)8, SM(PEG)12, SM(PEG)24, SPDP or SPDP, LC-SPDP and Sulfo-LC-SPDP, SMPT, PEG4-SPDP, PEG12-SPDP, SIA, SBAP, SIAB, Sulfo-SIAB, ANB-NOS, Sulfo-SANPAH, ATFB, SDA and Sulfo-SDA, LC-SDA and Sulfo-LC-SDA, SDAD and Sulfo-SDAD, DCC, EDC or EDAC, BMPH, EMCH, MPBH, KMUH, PDPH, PMPI, SPB.

Another aspect of the present invention provides a peptide construct of formula I

S₂—P—S₁—P (I)

wherein

P is a peptide consisting of the amino acid sequence X₁SRGGMRKX₂KKX₃X₄TX₅(SEQ ID NO: 1), wherein
X₁ is R or V,
X₂ is R or K, preferably X₂ is K
X₃ is P or K, preferably X₃ is K
X₄ is L or K, preferably X₄ is K
X₅ is G or K, preferably X₅ is K

S₂ is absent or is a spacer peptide sequence, having a length of at least 3 or at least 5 amino acid residues, or a polymer; preferably S₂ is absent or is a spacer peptide sequence selected from poly-Gly spacer, consisting of for example 3-16 glycines or 5-16 glycines, or Gly-rich spacer, preferably 3 to 16-amino acids Gly-rich spacer or 5 to 16-amino acids Gly-rich spacer; more preferably the spacer peptide sequence is selected from the group comprising GGGGSLVPRGSGGGGS (SEQ ID NO:10), GSGSGS (SEQ ID NO:11), GSGGGTGGGSG (SEQ ID NO:12), GGGGSGGGGS (SEQ ID NO:13), GGGGS (SEQ ID NO:14), GSGSGTGSGS (SEQ ID NO:15). In some embodiments, S₂ is absent or is a spacer peptide sequence or a polymer, wherein the spacer peptide sequence is a poly-Gly spacer consisting of 3-16 glycines or a 3 to 16-amino acids Gly-rich spacer; preferably the spacer peptide sequence is selected from the group comprising GGGGSLVPRGSGGGGS (SEQ ID NO:10), GSGSGS (SEQ ID NO:11), GSGGGTGGGSG (SEQ ID NO:12), GGGGSGGGGS (SEQ ID NO:13), GGGGS (SEQ ID NO:14), GSGSGTGSGS (SEQ ID NO:15).

S₁ is a spacer peptide sequence, having a length of at least 3 or at least 5 amino acid residues, or a polymer; preferably S₁ is a spacer peptide sequence selected from poly-Gly spacer, consisting of for example 3-16 glycines or 5-16 glycines, or Gly-rich spacer, preferably 3 to 16-amino acids Gly-rich spacer or 5 to 16-amino acids Gly-rich spacer; more preferably the spacer peptide sequence is selected from the group comprising GGGGSLVPRGSGGGGS (SEQ ID NO:10), GSGSGS (SEQ ID NO:11), GSGGGTGGGSG (SEQ ID NO:12), GGGGSGGGGS (SEQ ID NO:13), GGGGS (SEQ ID NO:14), GSGSGTGSGS (SEQ ID NO:15). In some embodiments, S₁ is a spacer peptide sequence or a polymer, wherein the spacer peptide sequence is a poly-Gly spacer consisting of 3-16 glycines or a 3 to 16-amino acids Gly-rich spacer; preferably the spacer peptide sequence is selected from the group comprising GGGGSLVPRGSGGGGS (SEQ ID NO:10), GSGSGS (SEQ ID NO:11), GSGGGTGGGSG (SEQ ID NO:12), GGGGSGGGGS (SEQ ID NO:13), GGGGS (SEQ ID NO:14), GSGSGTGSGS (SEQ ID NO:15).

In some embodiments of the peptide construct of the invention, P is a peptide selected from the group comprising:

(SEQ ID NO: 2)
RSRGGMRKRKKPLTG (IIa-8.0)
(SEQ ID NO: 3)
VSRGGMRKRKKPLTK (IIa-5.0)
(SEQ ID NO: 4)
VSRGGMRKRKKKKTG (IIa-7.1)
(SEQ ID NO: 5)
VSRGGMRKRKKPLTG (Ib-2.0)
(SEQ ID NO: 6)
VSRGGMRKKKKPLTG (Ib-1.0)
(SEQ ID NO: 7)
RSRGGMRKKKKPLTG (IIa-4.0)
(SEQ ID NO: 8)
VSRGGMRKKKKKKTG (IIa-3.1)
(SEQ ID NO: 9)
VSRGGMRKKKKPLTK (IIa-1.0)

In some embodiments of the peptide construct of the invention, P is RSRGGMRKRKKPLTG (SEQ ID NO:2) (IIa-8.0).

In some other embodiments of the peptide construct of the invention, P for each occurrence is same of different.

In some embodiments, P is a peptide that has the reversed amino acid sequence (reading from C- to N-terminus), namely X₅-T-X₄-X₃-K-K-X₂-KRMGGRS-X₁ (SEQ ID NO: 26).

In other embodiments of the peptide construct of the invention, the spacer is a polymer selected from the group comprising DSG, DSS, BS3, TSAT (trifunctional), BS(PEG)5, BS(PEG)9, DSP, DTSSP, DST, BSOCOES, EGS, Sulfo-EGS, DMA, DMP, DMS, DTBP, DFDNB, BMOE, BMB, BMH, TMEA (trifunctional), BM(PEG)2, BM(PEG)3, DTME, AMAS, BMPS, GMBS and Sulfo-GMBS, MBS and Sulfo-MBS, SMCC and Sulfo-SMCC, EMCS and Sulfo-EMCS, SMPB and Sulfo-SMPB, SMPH, LC-SMCC, Sulfo-KMUS, SM(PEG)2, SM(PEG)4, SM(PEG)6, SM(PEG)8, SM(PEG)12, SM(PEG)24, SPDP or SPDP, LC-SPDP and Sulfo-LC-SPDP, SMPT, PEG4-SPDP, PEG12-SPDP, SIA, SBAP, SLAB, Sulfo-SIAB, ANB-NOS, Sulfo-SANPAH, ATFB, SDA and Sulfo-SDA, LC-SDA and Sulfo-LC-SDA, SDAD and Sulfo-SDAD, DCC, EDC or EDAC, BMPH, EMCH, MPBH, KMUH, PDPH, PMPI, SPB.

The spacer described herein refers to a peptide sequence and/or a polymer that forms a flexible hinge separating the peptides “P” and thus allowing the peptides “P” of the peptide construct of formula I to be better recognized by the antiphospholipid antibodies (aPLA).

In some embodiments, the spacer peptide sequence can have a length of no more than 3, no more than 5, no more than 10, no more than 16, no more than 20, no more than 25, no more than 30, no more than 35, no more than 40, no more than 45, no more than 50, no more than 55, no more than 60, no more than 65, no more than 70, no more than 75, no more than 80, no more than 85, no more than 90, no more than 95 or no more than 100 amino acids. In some embodiments, the spacer peptide sequence can have a length of at least 3, at least 4, at least 5, at least 6, at least 7, at least 8, at least 9, at least 10, at least 12, at least 16, at least 18, at least 20, at least 25, at least 30, at least 35, at least 40, at least 45, or at least 50 amino acids. In some embodiments, the spacer peptide sequence comprises at least 3 and no more than 60 amino acids, at least 3 and no more than 55 amino acids, at least 3 and no more than 50 amino acids, at least 3 and no more than 45 amino acids, at least 3 and no more than 40 amino acids, at least 3 and no more 35 amino acids, at least 3 and no more than 30 amino acids, at least 3 and no more than 25 amino acids, at least 3 and no more than 20 amino acids or at least 3 and no more than 15 amino acids. In certain embodiments, the spacer peptide sequence comprises 3 to 20 amino acids, and in particular embodiments, comprises 6 to 16 amino acids or 5 to 16 amino acids.

The term “peptide” in the present invention designates a series of amino acid residues, typically L-amino acids, connected one to the other, typically by peptide bonds between the α-amino and carboxyl groups of adjacent amino acids.

An “immunogenic peptide”, “immunodominant epitope” or “peptide epitope” is a peptide which comprises an allele-specific motif or supermotif such that the peptide will bind an antiphospholipid antibodie (aPLA).

The peptides and the peptide constructs of the invention, such as the peptide construct of formula I, the isolated peptide or the peptide construct thereof, have been optimized which provides a sensitivity increase of the order of 200 times compared to the current techniques. This provides a simple, specific and reliable tool for quantification of circulating pathogenic antibodies, the number of false negatives could be reduced.

The present invention provides the opportunity to provide an accurate tool for the detection of aPLA, specifically β2GP1 antibodies for diagnostic purposes. Furthermore, aPLA-interacting motifs present in the peptides of the invention have the ability to inhibit aPLA activity and represent a prevention strategy for APS instead of anticoagulants. Finally, compositions containing the peptide constructs of the invention or the peptides of the invention associated with inducers of cell death can be used to specifically disrupt autoreactive T cells in APS patients, thus providing an excellent therapeutic approach.

In some embodiments of the invention, the peptides and the peptide constructs of the invention, such as the peptide construct of formula I, the isolated peptide or the peptide construct thereof, further comprise a binding moiety that connects said peptides and said peptide constructs of the invention to a solid surface, a solid support or a carrier molecule, such as a pharmaceutically acceptable carrier. The binding moiety can have the following functional groups selected from the group comprising —NH₂ functional group, —COOH functional group, —SH functional group, —CHO functional group, —OH functional group, and —N₃ functional group. In some embodiments of the invention, the binding moiety is selected from the group comprising amine, hydrazine, biotin, hydroxyl, avidin and aldehyde.

Peptides of the invention can be generated using recombinant DNA techniques, in bacteria, yeast, insect cells, plant cells or mammalian cells. Peptides of limited length can be prepared by chemical peptide synthesis, wherein peptides are prepared by coupling the different amino acids to each other. Chemical synthesis is particularly suitable for the inclusion of for example D-amino acids, amino acids with non-naturally occurring side chains or natural amino acids with modified side chains such as methylated cysteine.

Chemical peptide synthesis methods are well described, and peptides can be ordered from companies such as Applied Biosystems and other companies. Peptide synthesis can be performed as either solid phase peptide synthesis (SPPS) or contrary to solution phase peptide synthesis. The best-known SPPS methods are t-Boc and Fmoc solid phase chemistry. During peptide synthesis several protecting groups are used. For example, hydroxyl and carboxyl functionalities are protected by t-butyl group, Lysine and tryptophan are protected by t-Boc group, and asparagine, glutamine, cysteine and histidine are protected by trityl group, and arginine is protected by the pbf group. In particular embodiments, such protecting groups can be left on the peptide after synthesis.

Alternatively, the peptides can be synthesized by using nucleic acid molecules which encode the peptides of this invention in an appropriate expression vector which include the encoding nucleotide sequences. Such DNA molecules may be readily prepared using an automated DNA synthesizer and the well-known codon-amino acid relationship of the genetic code. Such a DNA molecule also may be obtained as genomic DNA or as cDNA using oligonucleotide probes and conventional hybridization methodologies. Such DNA molecules may be incorporated into expression vectors, including plasmids, which are adapted for the expression of the DNA and production of the polypeptide in a suitable host such as bacterium, for example Escherichia coli, yeast cell, animal cell or plant cell.

The physical and chemical properties of a peptide of interest (such as solubility, stability) are examined to determine whether the peptide is/would be suitable for use for applications as defined for the present invention. Typically this is optimised by adjusting the sequence of the peptide. Optionally, the peptide can be modified after synthesis (chemical modifications such as adding/deleting functional groups) using techniques known in the art.

Another aspect of the invention provides a pharmaceutical composition comprising the peptide construct of formula I of the invention in an amount effective to prevent, reduce or inhibit one or more symptoms of the antiphospholipid syndrome (APS) in a subject in need thereof, and a pharmaceutically acceptable carrier for administration of the peptide or the peptide construct and/or pharmaceutically acceptable excipients. Alternatively, the pharmaceutical composition of the invention comprises the isolated peptide or peptide construct thereof of the invention.

Pharmaceutical compositions for delivering peptides or peptide constructs are well known in the art. Such compositions typically contain drug carriers based on organic materials. In addition, different methods are known for polymer-peptide conjugation before being followed by physical encapsulation techniques, which is divided into surfactant-based techniques and polymer carriers. Surfactant-based techniques are dominated by liposome, microemulsions and solid-lipid nanoparticles. The field widens further in the polymer field. The delivery of peptides or peptide constructs has been enhanced using polymer-decorated liposomes, solid microspheres, polyelectrolyte complex, emulsions, hydrogels, and injectable polymers.

In some embodiments, the pharmaceutically acceptable carrier is the carrier molecule to which the peptides and the peptide constructs of the invention are optionally bound is selected from a wide variety of known carriers selected from the group comprising poly(sialic acid) (polysialylation), poly(glutamic acid) (glutamylation), homo-amino acid polymer (HAPylation), heparosan polymer (HEPylation), hydroxyethyl starch (HESylation), proline-alanine-serine repeats (PASylation) and unstructured polypeptides (XTENylation), erythrocytes/red blood cells (RBCs), OVA (Ovalbumin) human or bovine serum albumin, biotine and other polymers selected from the group comprising poly-aminoacids (e.g., polylysine), poly-esters (e.g., poly(lactic-co-glycolic) acid (PLGA), polylactic acid (PLA) and poly(ester amide)), polycaprolactone (PCL), polyanhydrides (e.g., poly(carboxyphenoxy propane-co-sebacic acid)) and carbohydrates (e.g., cyclodextrin). According to a particular embodiment, the carrier molecule is an amine-containing carrier protein. In one preferred embodiment, the peptides and the peptide constructs of the invention are covalently bound to the carrier molecule by its N-terminal end amino acid residue. In another preferred embodiment, the peptides and the peptide constructs of the invention are covalently bound to the carrier molecule by its C-terminal end amino acid residue. Also in some embodiments, the peptides and the peptide constructs of the invention are covalently bound to the carrier molecule through the binding moiety herein disclosed.

Molecules possessing a small size, that is, a low molecular mass, are rapidly cleared by renal filtration and degradation. With a growing number of therapeutics peptides being developed, many of them exhibiting a short plasma half-life, half-life extension strategies find increasing attention of biotech and pharmaceutical industries. Indeed, extension of the half-life can help to reduce the number of administrations (applications) and to lower doses, thus are beneficial for therapeutic but also economic reasons. The significant factors affecting half-life include the sequence of a peptide, modifications, administration routes, and the amount of the peptide (dose). It is observed that the sequence variants of a peptide have different half-lives. To achieve improved half-life, the inclusion of chemical modifications to the N-terminus or C-terminus of the peptides and the peptide constructs of the invention, such as pegylation, glycosylation, conjugation, Fc fusion, non-covalent interaction with serum albumin and covalent binding to albumin, is performed. Therefore, molecular size has a significant impact on clearance and half-life. Peptides <5 kDa are filtered very efficiently, and their clearance generally approaches the glomerular filtration rate. Because of the heterodimeric nature of these fusion proteins, the molecular mass is strongly increased resulting in a prolonged half-life.

In some embodiments of the invention, the peptides and the peptide constructs of the invention, such as the peptide construct of formula I, the isolated peptide or the peptide construct thereof, are bound to a pharmaceutically acceptable carrier, such as the carrier molecule herein disclosed through the binding moiety herein disclosed.

Another aspect of the invention provides a method for preventing and/or inhibiting one or more symptoms of the antiphospholipid syndrome (APS) in a subject comprising administering to said subject a therapeutically effect amount of the isolated peptide (or peptide construct thereof) of the invention, or the peptide construct of formula I of the invention or the pharmaceutical composition of the invention.

A “therapeutically effective amount” or “effective amount” of the peptide construct of formula I or the peptide of the present invention preferably results in a decrease in severity of disease symptoms, an increase in frequency and duration of disease symptom-free periods, an increase in lifespan, disease remission, or a prevention or reduction of impairment or disability due to the disease affliction. One of ordinary skill in the art would be able to determine a therapeutically effective amount based on such factors as the subject's size, the severity of the subject's symptoms, and the particular composition or route of administration selected.

A further aspect of the invention provides the isolated peptide (or peptide construct thereof) of the invention or the peptide construct of formula I of the invention for use in a method for preventing and/or inhibiting one or more symptoms of the antiphospholipid syndrome (APS).

The invention also provides a use of the peptide construct of formula I of the invention for the manufacturing of a medicament for treatment and/or prevention of the antiphospholipid syndrome (APS). Alternatively, the invention also provides use of the isolated peptide or peptide construct thereof of the invention for the manufacturing of a medicament for treatment and/or prevention of the antiphospholipid syndrome (APS).

Another aspect of the invention provides a use of the peptides and the peptide constructs of the invention, such as the peptide construct of formula I, the isolated peptide or the peptide construct thereof, for use as a pharmaceutical.

Another aspect of the invention provides a method for diagnosing of an antiphospholipid syndrome (APS) in a subject, wherein presence or absence of an antiphospholipid antibody (aPLA) is detected in a sample from the subject diagnosed, and wherein the presence of an antiphospholipid antibody is indicative of the APS disease and wherein the antiphospholipid antibody is detected using an immunoassay comprising the steps of
(i) providing a sample;
(ii) contacting the sample with the isolated peptide (or peptide construct thereof) of the invention or the peptide construct of formula I of the invention under conditions allowing for the formation of a complex between antiphospholipid antibodies with the isolated peptide (or peptide construct thereof) of the invention or the peptide construct of formula I of the invention;
(iii) detecting the complex.

In one embodiment, the invention also provides an isolated peptide (or peptide construct thereof) of the invention or a peptide construct of formula I of the invention for use in a method for diagnosing of an antiphospholipid syndrome (APS) in a subject, wherein presence or absence of an antiphospholipid antibody (aPLA) is detected in a sample from the subject diagnosed, and wherein the presence of an antiphospholipid antibody is indicative of the APS disease and wherein the antiphospholipid antibody is detected using an immunoassay comprising the steps of
(i) providing a sample;
(ii) contacting the sample with the isolated peptide (or peptide construct thereof) of the invention or the peptide construct of formula I of the invention under conditions allowing for the formation of a complex between antiphospholipid antibodies with the isolated peptide (or peptide construct thereof) of the invention or the peptide construct of formula I of the invention;
(iii) detecting the complex.

In another embodiment, the invention also provides a peptide (or peptide construct thereof) of the invention or a peptide construct of formula I of the invention for use in a method for diagnosing of an antiphospholipid syndrome (APS) in a subject, wherein presence or absence of an antiphospholipid antibody (aPLA) is detected in a sample from the subject diagnosed, and wherein the presence of an antiphospholipid antibody is indicative of the APS disease and wherein the antiphospholipid antibody is detected using an immunoassay comprising the steps of
(i) providing a sample;
(ii) contacting the sample with the peptide of any one of claims 1-2 immobilized on a surface or on beads or the peptide construct of any one of claims 3-4 immobilized on a surface or on beads, under conditions allowing for the formation of a complex between antiphospholipid antibodies with the peptide of any one of claims 1-2 or the peptide construct of any one of claims 3-4;
(iii) detecting the complex.

In an embodiment of the method for diagnosing of the invention, the peptide construct of formula I, or alternatively the isolated peptide or the peptide construct thereof, is immobilized on a surface or on beads. In another embodiment of the method for diagnosing of the invention, the complex is detected using a secondary antibody against the Fc portion of the antiphospholipid antibody, wherein preferably the antiphospholipid antibody is an IgG-antibody and/or the secondary antibody is an anti-IgG antibody, and/or the secondary antibody is preferably labelled with a detectable marker. In a further embodiment of the method for diagnosing of the invention, the complex is detected using a protein G that binds the Fc portion of the antiphospholipid antibody, wherein preferably the antiphospholipid antibody is an IgG-antibody and/or the protein G is preferably labelled with a detectable marker. In preferred embodiments, the immunoassay is selected from the group comprising ELISA, Lateral Flow Assay (LFA), immunoprecipitation, enzyme immunoassay (EIA), radioimmunoassay (MA), fluorescence immunoassay, a chemiluminescent assay, an agglutination assay, nephelometric assay, turbidimetric assay, a Western blot, a competitive immunoassay, a non-competitive immunoassay, a homogeneous immunoassay a heterogeneous immunoassay, a bioassay, and a reporter-assay such as a Luciferase-Assay of immunoprecipitation, enzyme immunoassay (EIA), radioimmunoassay (MA) or fluorescence immunoassay, a chemiluminescent assay, an agglutination assay, nephelometric assay, turbidimetric assay, a Western blot, a competitive immunoassay, a non-competitive immunoassay, a homogeneous immunoassay a heterogeneous immunoassay, a bioassay and a reporter-assay such as a Luciferase-Assay. Preferably, the immunoassay is an ELISA and/or Lateral Flow Assay (LFA).

Another aspect of the invention provides a method for detecting the presence of antiphospholipid antibody in a sample comprising
(i) providing a sample,
(i) contacting the sample with the isolated peptide (or peptide construct thereof) of the invention or the peptide construct of formula I of the invention under conditions allowing for the formation of a complex between antiphospholipid antibodies with the isolated peptide (or peptide construct thereof) of the invention or the peptide construct of formula I of the invention;
(iii) detecting the complex using an immunoassay.

In one embodiment, the invention also provides a method for detecting the presence of antiphospholipid antibody in a sample comprising
(i) providing a sample,
(i) contacting the sample with the peptide of any one of claims 1-2 immobilized on a surface or on beads or the peptide construct of any one of claims 3-4 immobilized on a surface or on beads, under conditions allowing for the formation of a complex between antiphospholipid antibodies with the peptide of any one of claims 1-2 or the peptide construct of any one of claims 3-4;
(iii) detecting the complex using an immunoassay.

In an embodiment of method for detecting the presence of antiphospholipid antibody in a sample of the invention, the peptide construct of formula I, or alternatively the isolated peptide or the peptide construct thereof, is immobilized on a surface or on beads. In another embodiment of the method for detecting the presence of antiphospholipid antibody in a sample of the invention, the complex is detected using a secondary antibody against the Fc portion of the antiphospholipid, wherein preferably the antiphospholipid antibody is an IgG-antibody and the secondary antibody is an anti-IgG antibody, and/or the secondary antibody is preferably labelled with a detectable marker. In a further embodiment of the method for detecting the presence of antiphospholipid antibody in a sample of the invention, the complex is detected using a protein G that binds the Fc portion of the antiphospholipid antibody, wherein preferably the antiphospholipid antibody is an IgG-antibody and/or the protein G is preferably labelled with a detectable marker. In preferred embodiments, the immunoassay is selected from the group comprising ELISA, Lateral Flow Assay (LFA), immunoprecipitation, enzyme immunoassay (EIA), radioimmunoassay (MA), fluorescence immunoassay, a chemiluminescent assay, an agglutination assay, nephelometric assay, turbidimetric assay, a Western blot, a competitive immunoassay, a non-competitive immunoassay, a homogeneous immunoassay a heterogeneous immunoassay, a bioassay, and a reporter-assay such as a Luciferase-Assay. Preferably, the immunoassay is ELISA and/or Lateral Flow Assay (LFA).

The invention also encompasses a diagnostic immunoassay for determining the presence of aPL antibody in a sample (such as body fluids) taken from subjects suspected of suffering from an aPL antibody-mediated disease comprising contacting a sample of a body fluid with peptides or peptide constructs of the invention, such as the peptide construct of formula I, the isolated peptide or the peptide construct thereof of the invention, which specifically binds aPL antibodies and determining by methods well known in the art whether aPL antibodies are present in the sample and, if present, quantitating the amount of aPL antibodies present in the sample. One such immunoassay comprises: (a) coating wells of a microtitration plate with a peptide or a peptide construct of the invention, such as the peptide construct of formula I, the isolated peptide or the peptide construct thereof of the invention, which specifically binds aPL antibodies; (b) washing the wells to wash away unbound peptide or peptide construct; (c) adding a test sample of a sample obtained from a subject to the wells wash away unbound peptide or peptide construct; (d) adding a test sample of a sample obtained from a subject to the wells and incubating for a pre-determined time; (e) washing the wells to remove unbound test sample; (f) adding anti-human IgG conjugated with a label to the wells of the plate and incubating for a pre-determined time; (g) washing the wells to wash away unbound anti-human IgG conjugate; (h) adding a substrate for the labelled conjugate and developing the substrate/label reaction for a pre-determined time; (i) measuring the end-product of the substrate/label reaction to determine the presence of anti-aPL antibody in the test sample. A diagnostic immunoassay as described above wherein the immunoassay is quantitative is also encompassed.

Another aspect of the invention provides a use of the peptides and the peptide constructs of the invention, such as the peptide construct of formula I, the isolated peptide or the peptide construct thereof of the invention, for detecting antiphospholipid antibodies.

In some embodiments of the invention, the antiphospholipid antibodies are IgG anti-β2-glycoprotein-1 (aPL) antibodies.

A sample for use in the methods for diagnosing of the invention may be derived from different sources. It is understood that a “sample” as contemplated herein includes also a sample that is modified from its original state, for example, by purification, dilution or the addition of any other component or components, such as the addition of chemical or biochemical substances to the solution, such as acids, bases, buffers, salts, solvents, reactive dyes, detergents, emulsifiers, chelators. The sample is preferably a biological sample, such as body fluid sample. Non-limiting examples of biological samples include whole blood or a component thereof (e.g. plasma, serum), urine, saliva lymph, bile fluid, sputum, tears, cerebrospinal fluid, bronchioalveolar lavage fluid, synovial fluid, semen, ascitic tumour fluid, breast milk and pus. In preferred embodiments, the sample is blood sample, plasma sample and/or serum sample. The biological sample may be derived from a healthy individual, or an individual suffering from a particular disease or condition, such as antiphospholipid syndrome (APS). For example, the individual may be suffering from or suspected to be suffering from an autoimmune disease, such as antiphospholipid syndrome (APS). The biological sample may be collected from a subject and used directly. Alternatively, the biological sample may be processed prior to use. For example, the biological sample may be purified, concentrated, separated into various components, or otherwise modified prior to use. It will be understood that a biological sample as contemplated herein includes cultured biological materials, including a sample derived from cultured cells, such as culture medium collected from cultured cells or a cell pellet. Accordingly, a biological sample may refer to a lysate, homogenate or extract prepared from a whole organism or a subset of its tissues, cells or component parts, or a fraction or portion thereof. A biological sample may also be modified prior to use, for example, by purification of one or more components, dilution, and/or centrifugation.

Another aspect of the invention provides an antibody or an antigen-binding fragment thereof, that binds to the peptide comprising or consisting of the sequence defined by SEQ ID NO: 1 or binds to the peptide construct of formula I.

In an embodiment of the invention, the antibody that binds to the peptide comprising or consisting of the sequence defined by SEQ ID NO: 1 or to the peptide construct of formula I is a monoclonal antibody.

In an embodiment of the invention, the antibody that binds to the peptide comprising or consisting of the sequence defined by SEQ ID NO: 1 or binds to the peptide construct of formula I comprises a heavy chain variable region that comprises CDR1, CDR2, and CDR3 domains; and a light chain variable region that comprises CDR1, CDR2, and CDR3 domains, wherein the heavy chain variable region CDR1, CDR2, and CDR3 sequences are as set forth in SEQ ID NO: 16, SEQ ID NO: 17, and SEQ ID NO: 18, respectively and the light chain variable region CDR1, CDR2, and CDR3 sequences are as set forth in SEQ ID NO: 19, SEQ ID NO: 20, and SEQ ID NO: 21, respectively.

Heavy chain CDR1:
(SEQ ID NO: 16)
SYWIQ
Heavy chain CDR2:
(SEQ ID NO: 17)
AIYPGDGDTSYTQKFKG
Heavy chain CDR3:
(SEQ ID NO: 18)
LGDGYDDYAMDY
Light chain CDR1:
(SEQ ID NO: 19)
RASESVDSYGNSFMH
Light chain CDR2:
(SEQ ID NO: 20)
LASNLES
Light chain CDR3:
(SEQ ID NO: 21)
QQNNEDPYT

In other embodiment of the invention, the antibody that binds to the peptide consisting of the sequence defined by SEQ ID NO: 1 or to the peptide construct of formula I comprises a heavy chain variable region comprising the amino acid sequence as set forth in SEQ ID NO: 22 and a light chain variable region comprising the amino acids sequence as set forth in SEQ ID NO: 23.

Heavy chain:
(SEQ ID NO: 22)
QVQLQQSGAELARPGASVKLSCKASGYTFSSYWIQWVKQRPGQGLEWI
GAIYPGDGDTSYTQKFKGKATLTADKSSSTAYMQLSSLASEDSAVYYC
ARLGDGYDDYAMDYWGQGTSVTVSS
Light chain:
(SEQ ID NO: 23)
NIVLTQSPASLAVSLGQRATISCRASESVDSYGNSFMHWYQQKPGQPP
KLLIYLASNLESGVPARFSGSGSRTDFTLTIDPVEADDVATYYCQQNN
EDPYTFGGGTKLEIK

In another aspect, the antibody, or an antigen-binding fragment thereof, of the invention comprises a heavy chain variable region (VH) sequence and/or a light chain variable region (VL) sequence having at least 90%, 91%, 92%, 93%, 94%, 95%, 96%, 97%, 98%, 99%, or 100% sequence identity to the amino acid sequence of SEQ ID NO: 22 and/or SEQ ID NO: 23. In certain embodiments, a VH sequence and/or VL sequence having at least 90%, 91%, 92%, 93%, 94%, 95%, 96%, 97%, 98%, or 99% identity contains substitutions (such as conservative substitutions), insertions, or deletions relative to the reference sequence, but an antibody, or an antigen-binding fragment thereof, comprising that sequence retains the ability to bind to the peptide consisting of the sequence defined by SEQ ID NO: 1 or to the peptide construct of formula I. In certain embodiments, a total of 1 to 10 amino acids have been substituted, inserted and/or deleted in SEQ ID NO: 22 and/or in SEQ ID NO: 23. In certain embodiments, substitutions, insertions, or deletions occur in regions outside the HVRs (for example in the FRs). Optionally, the antibody, or an antigen-binding fragment thereof, comprises the VH sequence and/or VL sequences SEQ ID NO: 22 and/or SEQ ID NO: 23, including post-translational modifications of that sequence.

In another aspect, the antibody, or an antigen-binding fragment thereof, of the invention comprises a heavy chain variable region that comprises CDR1, CDR2, and CDR3 domains sequences and/or a light chain variable region that comprises CDR1, CDR2, and CDR3 domains sequences having at least 90%, 91%, 92%, 93%, 94%, 95%, 96%, 97%, 98%, 99%, or 100% sequence identity to the amino acid sequence of one or more SEQ ID NOs: 16 to 21. In certain embodiments, the CDR domains sequences having at least 90%, 91%, 92%, 93%, 94%, 95%, 96%, 97%, 98%, or 99% identity contains substitutions (such as conservative substitutions), insertions, or deletions relative to the reference sequence, but an antibody, or an antigen-binding fragment thereof, comprising that sequence retains the ability to bind to the peptide consisting of the sequence defined by SEQ ID NO: 1 or to the peptide construct of formula I. In certain embodiments, a total of 1 to 10 amino acids have been substituted, inserted and/or deleted in one or more SEQ ID NOs: 16 to 21. In certain embodiments, substitutions, insertions, or deletions occur in regions outside the HVRs (for example in the FRs). Optionally, the antibody, or an antigen-binding fragment thereof, comprises the CDR domains sequences SEQ ID NOs: 16 to 21, including post-translational modifications of that sequence.

According to an embodiment of the invention, the antigen-binding fragment of the antibody of the invention is a minibody that binds to the same epitope as antiphospholipid antibodies. In an embodiment, the minibody binds an epitope consisting of the sequence defined by SEQ ID NO: 2 or SEQ ID NO: 1. In an embodiment, said minibody comprises a variable heavy chain fragment, such as a heavy chain fragment as set forth in SEQ ID NO:22, a variable light chain fragment, such as a light chain fragment as set forth in SEQ ID NO:23, and a hinge domain between the variable light chain fragment and the constant chain fragment. In another embodiment, said minibody comprises a heavy chain variable region that comprises CDR1, CDR2, and CDR3 domains; and a light chain variable region that comprises CDR1, CDR2, and CDR3 domains, wherein the heavy chain variable region CDR1, CDR2, and CDR3 sequences are as set forth in SEQ ID NO: 16, SEQ ID NO: 17, and SEQ ID NO: 18, respectively and the light chain variable region CDR1, CDR2, and CDR3 sequences are as set forth in SEQ ID NO: 19, SEQ ID NO: 20, and SEQ ID NO: 21, respectively. The minibody of the invention is typically used as a specific aPL neutralizing therapy by indirect competitive inhibition.

The invention also provides methods of producing the antibodies, or the antigen-binding fragments thereof, of the invention using recombinant techniques. For example, polypeptides can be prepared using isolated nucleic acids encoding such antibodies or fragments thereof, vectors and host-cells comprising such nucleic acids.

An aspect of the present invention provides an isolated nucleic acid encoding the antibody, or an antigen-binding fragment thereof, of the invention.

Another aspect of the present invention provides a vector comprising a nucleic acid encoding the antibody, or an antigen-binding fragment thereof, of the invention. In an embodiment, the vector of the invention is an expression vector.

Another aspect of the present invention provides a host cell comprising a nucleic acid encoding the antibody, or an antigen-binding fragment thereof, of the invention or comprising the vector of the invention. In an embodiment, the host cell of the invention is prokaryotic or eukaryotic.

Antibodies may be produced using recombinant methods and compositions, such as described in U.S. Pat. No. 4,816,567. In one embodiment, isolated nucleic acid encoding an antibody of the invention is provided. Such nucleic acid may encode an amino acid sequence comprising the VL and/or an amino acid sequence comprising the VH of the antibody (e.g., the light and/or heavy chains of the antibody). In some embodiments, the isolated nucleic acid encodes a VH amino acid sequence consisting of SEQ ID NO: 22. In some embodiments, the isolated nucleic acid encodes a VL amino acid sequence consisting of SEQ ID NO: 23.

For recombinant production of antibodies, or antigen-binding fragments thereof, of the invention, nucleic acids encoding the desired antibodies or antibody fragments of the invention, are isolated and inserted into a replicable vector for further cloning (amplification of the DNA) or for expression. In a further embodiment, one or more vectors (such as expression vectors) comprising such nucleic acid are provided. In some embodiments, a vector comprises a nucleic acid encoding a VH amino acid sequence consisting of SEQ ID NO: 22. In some embodiments, a vector comprises a nucleic acid encoding a VL amino acid sequence consisting of SEQ ID NO: 23. DNA encoding the polyclonal or monoclonal antibodies is readily isolated (for example, with oligonucleotide probes that specifically bind to genes encoding the heavy and light chains of the antibody) and sequenced using conventional procedures. Many cloning and/or expression vectors are commercially available. Vector components generally include, but are not limited to, one or more of the following, a signal sequence, an origin of replication, one or more marker genes, a multiple cloning site containing recognition sequences for numerous restriction endonucleases, an enhancer element, a promoter, and a transcription termination sequence.

Another aspect of the present invention provides the antibody, or an antigen-binding fragment thereof, of the invention for use as a pharmaceutical. In one embodiment, the antigen-binding fragment is a minibody, for example the minibody of the invention.

Another aspect of the invention provides a method for preventing and/or inhibiting one or more symptoms of the antiphospholipid syndrome (APS) in a subject comprising administering to said subject a therapeutically effect amount of the antibody, or an antigen-binding fragment thereof, of the invention.

A “therapeutically effective amount” or “effective amount” of the antibody, or an antigen-binding fragment thereof, of the invention preferably results in a decrease in severity of disease symptoms, an increase in frequency and duration of disease symptom-free periods, an increase in lifespan, disease remission, or a prevention or reduction of impairment or disability due to the disease affliction. One of ordinary skill in the art would be able to determine a therapeutically effective amount based on such factors as the subject's size, the severity of the subject's symptoms, and the particular composition or route of administration selected.

A further aspect of the invention provides the antibody, or an antigen-binding fragment thereof, of the invention for use in a method for preventing and/or inhibiting one or more symptoms of the antiphospholipid syndrome (APS).

The invention also provides a use of the antibody, or an antigen-binding fragment thereof, of the invention for the manufacturing of a medicament for treatment and/or prevention of the antiphospholipid syndrome (APS).

Another aspect of the invention provides a kit for detecting in a sample the presence or absence of an antiphospholipid antibody, the kit comprising the peptide construct of formula I of the invention or the isolated peptide or peptide construct thereof of the invention. The kit also comprises the at least the instructions for use. The kit may also further comprise at least one antibody of the invention. Such antibody can be used as calibrating antibody (calibrator).

Kits of the invention may include other components required to conduct the methods of the present invention, such as buffers and/or diluents. The kits may comprise one or more means for obtaining a sample from a subject. The kits typically include containers for housing the various components and/or instructions for using the kit components in the methods of the invention. Kits of the invention may comprise a suitable support on which one or more reagents are immobilised or may be immobilised, for example, kits of the invention may comprise a support coated with a peptide construct of formula I of the invention, an isolated peptide or a peptide construct thereof, an antibody, streptavidin, or biotin. Non-limiting examples of suitable supports include assay plates (e.g. micro titer plates) or test tubes or beads manufactured from polyethylene, polypropylene, polystyrene, Sephadex, polyvinyl chloride, plastic beads, and, as well as particulate materials such as filter paper, nitrocellulose membrane, agarose, cross-linked dextran, and other polysaccharides.

Kits of the invention may be used to perform an enzyme-linked immunosorbent assay (ELISA) and/or Lateral Flow Assay (LFA). Additionally or alternatively, kits of the invention may be used to perform western blotting. Such kits may further comprise a carrier, package or container that is compartmentalized to receive one or more containers such as vials, tubes, and the like, each of the container(s) comprising one of the separate elements to be used in the method. The kits of the invention will typically comprise the container comprising the elements described above and one or more other containers comprising materials desirable from a commercial and user standpoint, including buffers, diluents, filters, needles, syringes, and package inserts with instructions for use. In addition, a label can be provided on the container to indicate that the composition is used for a specific therapeutic or non-therapeutic application, and can also indicate directions for either in vivo or in vitro use, such as those described herein. Directions and or other information can also be included on an insert which is included with the kits. The kits preferably comprises means for handling and/or processing a blood sample.

A further aspect of the present invention provides a device for detecting in a sample the presence or absence of an antiphospholipid antibody, the device comprising a solid support comprising the peptide construct of formula I of the invention or the isolated peptide or peptide construct thereof of the invention.

The peptides and the peptide constructs of the invention, such as the peptide construct of formula I, the isolated peptide or the peptide construct thereof of the invention, are particularly useful in ELISA and/or Lateral Flow Assay (LFA). The device typically has a housing comprising a solid support to which the peptide construct of formula I of the invention, or the isolated peptide or the peptide construct thereof of the invention, is bound (i.e. provides coated solid support). Acceptable materials for the device housing include water impermeable plastics such as polystyrene, polypropylene, polyvinyl chloride and the like. The solid support can be of any suitable material, such as plastics, gold, silica, or silicon.

Another aspect of the present invention provides an immunoaffinity membrane comprising the peptide construct of formula I of the invention or the isolated peptide or peptide construct thereof of the invention.

In one embodiment, the immunoaffinity membrane comprises a microporous membrane. In another embodiment, the immunoaffinity membrane comprises a dialysis or ultrafiltration membrane. In a further embodiment, the immunoaffinity membrane comprises a hollow fiber or a flat sheet. In another embodiment, the immunoaffinity membrane is comprised of an organic polymer or an inorganic material to which the peptide construct of formula I of the invention, or the isolated peptide or the peptide construct thereof, can be attached. In another embodiment, the immunoaffinity membrane is comprised of a material selected from the group consisting of nylon, polysulfone, cellulose triacetate, cuprophane, ethylene vinyl alcohol polymers, or ethylene vinyl alcohol copolymer (EVAL). In another embodiment, the immunoaffinity membrane has minimal nonspecific binding to blood components other than antiphospholipid antibodies.

Another aspect of the invention provides a method of selectively removing antiphospholipid antibodies from blood, serum or plasma comprising the steps of immobilizing a ligand capable of binding to an antiphospholipid antibody present in blood, serum or plasma to an immunoaffinity membrane and passing blood, serum or plasma through said immunoaffinity membrane so that antiphospholipid antibodies from the blood, serum or plasma will be removed by the immunoaffinity membrane, wherein the ligand is the peptide construct of formula I of the invention or an isolated peptide or peptide construct thereof of the invention.

In an embodiment, the invention provides the peptide construct of formula I of the invention or the isolated peptide or the peptide construct thereof of the invention for use in a method of selectively removing antiphospholipid antibodies from blood, serum or plasma comprising the steps of immobilizing the peptide construct of formula I of the invention or the isolated peptide or the peptide construct thereof of the invention to an immunoaffinity membrane and passing blood, serum or plasma through said immunoaffinity membrane so that antiphospholipid antibodies from the blood, serum or plasma will be removed by the immunoaffinity membrane.

In another embodiment, the method of selectively removing antiphospholipid antibodies from blood, serum or plasma invention is performed continuously using a single apparatus.

In another embodiment, in the method of selectively removing antiphospholipid antibodies from blood, serum or plasma, the blood, serum or plasma is introduced to the membrane extracorporeally, and wherein the blood, serum or plasma is collected following removal of the antiphospholipid antibodies and reintroduced into a patient.

Another aspect of the present invention provides an apparatus suitable for performing the method of selectively removing antiphospholipid antibodies from blood, serum or plasma comprising an immunoaffinity membrane having a ligand immobilized thereto capable of binding to an antiphospholipid antibody and a device for passing blood, serum or plasma through said immunoaffinity membrane, wherein the ligand is the peptide construct of formula I of the invention or the isolated peptide or peptide construct thereof of the invention.

In an embodiment of the apparatus of the invention, the device for passing blood, serum or plasma through the immunoaffinity membrane comprises a single piece of equipment. Any suitable device known in the art, such as pumps, plasma pumps or equivalent, can be used in the method of the invention.

Another aspect of the invention provides a cartridge comprising the immunoaffinity membrane of the invention or the peptide construct of formula I of the invention or the isolated peptide or peptide construct thereof of the invention. The cartridge is typically used the method of selectively removing antiphospholipid antibodies from blood, serum or plasma or in the apparatus suitable for performing the method of selectively removing antiphospholipid antibodies from blood, serum or plasma.

Those skilled in the art will appreciate that the invention described herein is susceptible to variations and modifications other than those specifically described. It is to be understood that the invention includes all such variations and modifications without departing from the spirit or essential characteristics thereof. The invention also includes all of the steps, features, compositions and compounds referred to or indicated in this specification, individually or collectively, and any and all combinations or any two or more of said steps or features. The present disclosure is therefore to be considered as in all aspects illustrated and not restrictive, the scope of the invention being indicated by the appended claims, and all changes which come within the meaning and range of equivalency are intended to be embraced therein.

The foregoing description will be more fully understood with reference to the following Examples. Such Examples, are, however, exemplary of methods of practising the present invention and are not intended to limit the application and the scope of the invention.

### Methods

### Examples

Ethical Statement

All breeding and experimental protocols and procedures were reviewed and approved by the Institutional Animal Care and Use Committee of the Geneva University School of Medicine. Animal care and experimental procedures were carried out in accordance with the guidelines of the Institutional Animal Care and Use Committee of the Geneva University School of Medicine and complied with the guidelines from Directive 2010/63/EU of the European Parliament on the protection of animals used for scientific purposes.

Patient Characteristics

All patients had an APS, as defined by the revised Sapporo criteria. Blood was obtained from each patient with written consent and approval by the institutional ethics committee of the University Hospital of Geneva, in accordance with the Helsinki declaration. The characteristics of the patients used in this study have been already presented in previous publications^{1.6}.

Study Population and Clinical Assessment

This cohort of patient has been already used for previous publication.⁷ Briefly, this retrospective study was conducted between October 2012 and November 2014 among 380 consecutive outpatient pregnant women aged 18 or older attending the Diabetology Unit of IRCCS Ospedale Policlinico San Martino (Genoa, Italy) to perform a 75-g oral glucose tolerance test (OGTT) for the screening of gestational diabetes (GDM) The Ethics Committee of IRCCS Ospedale Policlinico San Martino in Genoa (Italy) approved this protocol, performed in accordance with the guidelines of the Declaration of Helsinki. Patients gave written informed consent before entering the study.

Mice

B6.Nba2. Yaa mice were generated as described.⁸ The Apoe^{-/-} null mutation was introduced in B6.Nba2.Yaa mice by breeding. Eleven-week old Apoe^{-/-} C57B1/6 and Apoe^{-/-}Nba2.Yaa mice were subjected to 11 weeks of high cholesterol diet (HCD) (20.1% fat, 1.25% cholesterol, Research Diets, Inc., New Brunswick, NJ), as a model of advanced atherosclerosis. The treatments and atherosclerosis protocols were well-tolerated by the mice, and no adverse events (such as weight loss and signs of systemic toxicity) were reported. At sacrifice, haematological parameters were routinely measured. Animals were euthanized by exsanguination after anesthesia with 4% isoflurane. All breeding and experimental protocols and procedures were reviewed and approved by the Institutional Animal Care and Use Committee of the Geneva University School of Medicine.

Microarray

The peptide microarray have been perform blinded by PEPperPRINT GmbH, Heidelberg, as follow: Pre-staining of a peptide microarray was done with secondary goat anti-human IgG (H+L) DyLight680 antibody (1:5000) and control mouse monoclonal anti-HA (12CA5) DyLight800 antibody (1:2000) to investigate background interactions with the variants of wild type peptide that could interfere with the main assays. Subsequent incubation of other peptide microarray copies with human antibodies at concentrations of 100 μ/ml and 500 μg/ml in incubation buffer was followed by staining with secondary and control antibodies as well as read-out at scanning intensities of 7/7 (red/green). The control staining of the HA epitopes was done as internal quality control to confirm the assay quality and the peptide microarray integrity. Quantification of spot intensities and peptide annotation were based on the 16-bit gray scale tiff files at scanning intensities of 7/7 that exhibit a higher dynamic range than the 24-bit colorized tiff files; microarray image analysis was done with PepSlide® Analyzer. A software algorithm breaks down fluorescence intensities of each spot into raw, foreground and background signal, and calculates averaged median foreground intensities and spot-to-spot deviations of spot triplicates. Based on averaged median foreground intensities, an intensity map was generated and interactions in the peptide map highlighted by an intensity color code with red for high and white for low spot intensities. It was tolerated a maximum spot-to-spot deviation of 40%, otherwise the corresponding intensity value was zeroed.

Immunoassays

Determination of aPL by ELISA—MaxiSorp™ 96 well plates (Nunc) or Streptavidin plate (Thermofisher) were coated with 10 μg/ml recombinant domains of β2GP1, peptides or biotinylated-peptide prior to incubation with aPLA. Secondary anti-human antibodies conjugated to IR800CW (Rockland) or HRP were used. Protein- or peptide-bound antibodies were detected and quantified by the Odyssey system (Li-Cor Biosciences) or absorbance in optical densities (OD) was determined at 405 nm (molecular Devices™ Filtermax).

Determination of autoantibodies anti-apoA-1 by ELISA—Maxisorp plates (Nunc™, Denmark) were coated with purified, derived delipidated murine recombinant apolipoprotein A-1 (Biorbyt, United Kingdom) (20 mg/ml; 50 ml/well) for 1 h at 37° C. After washing, all wells were blocked for 1 h with 2% bovine serum albumin (BSA) in phosphate buffer solution (PBS) at 37° C. Then, samples were incubated for 1 h. Samples were also added to a non-coated well to assess individual non-specific binding. After washing 50 μl/well of signal antibody (alkaline phosphatase-conjugated anti-human IgG; Sigma-Aldrich, St Louis, MO) dilute 1:1000 in PBS/BSA 2% solution was incubated 1 h at 37° C. After washing, phosphatase substrate p-nitrophanyl phosphate disodium (Sigma-Aldrich) dissolved in diethanolamine buffer (pH 9.8) was added. Each sample was tested in duplicate and absorbance in optical densities (OD) was determined at 405 nm after 20 min of incubation at 37° C. (molecular Devices™ Filtermax). Corresponding non-specific binding was subtracted from mean absorbance for each sample. Determination of autoantibodies anti-dsDNA by ELISA—Salmon Sperm dsDNA was coated to ELISA plates precoated with poly L lysine (Sigma-Aldrich). Plates were then incubated with 1/100 diluted serum samples, and development performed with alkaline phosphatase-labelled goat anti-mouse IgM or IgG. Results are expressed in U/mL in reference to a standard curve.

Surface Plasmon Resonance (SRP)

The kinetics and affinity of protein-protein and protein-lipid interactions were determined using a BIAcore X100 instrument. 1 mg/ml of biotin-tagged peptide (ligand) was immobilized using a sensor chip SA (GE Healthcare) surface, whereas aPL from a pool of 13 Human patients were flown as analyte. The first flow cell of the sensor chip was used as a control surface (no protein), whereas the second flow cell was employed as the active surface. A range of dilution of aPL analytes prepared in the same buffer was injected on both flow cell surfaces at a flow rate of 30 μl/min. Association and dissociation times for each protein injection were set at 90 and 120 s, respectively. In all cases, sensorgrams were obtained from three different dilutions of aPL.

Statistical Analysis

Statistics were performed using GraphPad Prism 8, Statistica (version 13.0). Data are presented as mean±SEM. For clinical scores, significance between groups was analyzed using the nonparametric Mann-Whitney U test. Spearman's rank correlation coefficients were used to assess correlations between variables. The number of mice used for each analysis is indicated in the figure legends. All data are presented as the mean±SEM and the statistical significance threshold used is *: p≤0.05. **: p≤0.05; ***: p≤0.005.

Epitope R39-R43 is Only a Part of the Epitope-Determining Sequence for aPL

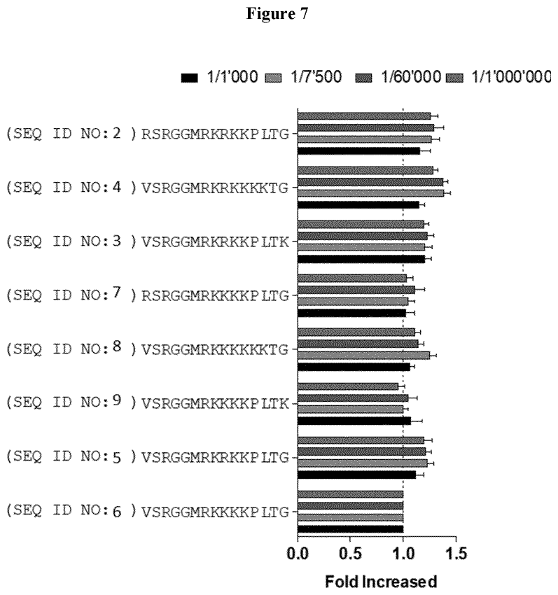

The inventors performed a peptide substitution scan of wild type peptide VSRGGMRKFICPLTG (SEQ ID NO: 24) carrying the epitope R39-R43 and based on an exchange of the underlined amino acid positions with the 20 main amino acids. The resulting peptide microarrays contained 136 different peptides. It was observed that the peptide substitution for the position n° 4 to 10 (not shown) have no real influences on its ability to interact with aPL neither from patients 1, 2, 3, 4 nor the pool of plasma. However, the substitution of the position 11 by an Arginine (R) and, in particular, a Lysine (K) increases the affinity of the peptide for four aPL and the pool of patients (not shown). While the substitution of the position n° 11 by a Lys shows a stronger interaction with aPL, it was decided to perform a second peptide substitution scan on the VSRGGMRKFIKPLTG (SEQ ID NO: 25) based on an exchange of the underlined amino acid positions with the 20 main amino acids to identify a potential additional improvement of the affinity for aPL. The microarrays contained 400 different peptides. Within this library, it can be noticed that the residue present in position n° 9 has a strong influence on the affinity of aPL. Although the presence of a Lysine or Arginine in position n° 9 with different residues in position n° 10 could have some positive effects on the interaction with aPL, the association of Lysine and Arginine to position n° 9 and 10 leads to a robust binding to aPL. It appears, however, that the combination of two Lysines in position n° 9 and 10 have the strongest affinity for aPL (not shown). These results demonstrate that the core sequence of the β2GP1-derived peptide embedded R39-R43 epitope and 4 Lysines next to Arginine 43. See also FIGS. 6 and 7.

Epitope Recognized by aPL Requires to be Spatially Oriented for Optimal Interactions

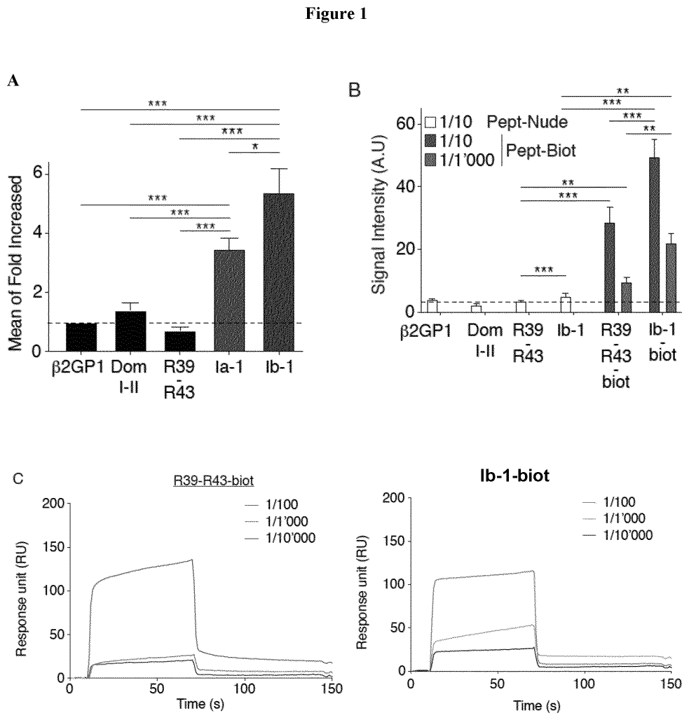

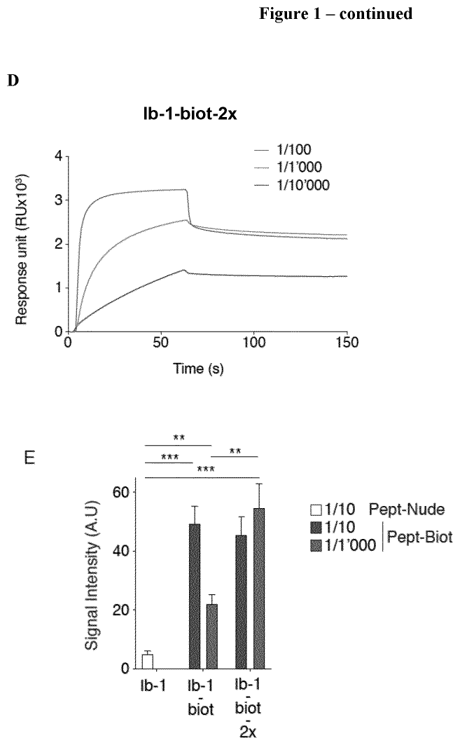

In order to compare the ability of β2GP1, domain I-II of β2GP1, R39-R43 peptide (VSRGGMRKFICPLTG (SEQ ID NO: 24)), Ia-1 peptide (VSRGGMRKFIKPLTG (SEQ ID NO: 25); corresponding to the first substitution scan (not shown) and Ib-1 peptide; corresponding to the second substitution scan (not shown) to bind to aPL, custom immunoassays were performed. The binding level of aPL to (β2GP1 was taken as reference. It could be seen that Dom MI and R39-R43 has the same ability to interact with aPL than β2GP1 while the interaction of Ia-1 and Ib-1 peptide have an increase fold mean of 3.47 and 5.5 time, respectively (FIG. 1A). The de Groot's research group has pointed at the importance of hydrophobic character of the plate during coating of R39-R43 epitope. In order to abrogate of the plate-dependent aPL binding, the Ia-1 and Ib-1 peptides as well as the R39-R43 with a biotin at their N-terminal were synthetized and coated a streptavidin plate. In this configuration, the different peptides are flag oriented in the space preventing any interaction with the plate. It was thus observed that the flag-type orientation of R39-R43-biot leads to 8.52×more interaction with aPL while they show 9.63×more binding for Ib-1-biot presenting the same position. The interactions still remain 2.83× and 4.3× with R39-R43-biot and Ib-1-biot, respectively, after a dilution of 100× of aPL (FIG. 1B). The surface plasmon resonance (SPR) technique is used to determine the relative avidity of aPL for R39-R43-biot and Ib-1-biot. The interaction between aPL and immobilized R39-R43-biot or Ib-1-biot is monitored by flowing various concentrations of aPL over a R39-R43-biot- or Ib-1-biot-coated chip surface (FIG. 1C). Through SPR experiments, it appears that aPL avidity for Ib-1-biot presents a resonance unit (RU) of 51.49 at 1/1,000 of dilution and a RU of 27.61 at 1/10,000 of dilution while, at the same dilution, it present a RU of 27.36 and a RU of 17.99 with R39-R43-biot, respectively (FIG. 1C). However, at higher concentrations, the peptides seems to show no differences in the interactions with aPL. It could be further observed that neither R39-R43-biot nor Ib-1-biot have sustained interaction with aPL considering the stabilization curves. Considering that IgG valency, it was decided to generate a dimeric Ib-1-biot peptide (Ib-1-biot-2x). This new polypeptide carries thus two optimized epitopes which have the opportunity to interact with two Fab fragment present on aPL. SPR experiments performed with Ib-1-biot-2x-coated chip surface show that the avidity of aPL for is 47.8× and 48.9×more at dilution of 1/1,000 and 1/10,000, respectively, than the avidity for Ib-1-biot (FIG. 1D). At higher concentration, i.e dilution of 1/100, the avidity for dimeric peptide is 28×more than for the monomeric (FIG. 1D). While the association of two epitopes leads to stronger interaction, the sustained stability of binding is also significantly increased as it could be observed through the shape of the curves (FIG. 1D). The dimeric peptide Ib-1-biot-2x shows thus a stronger ability to retain aPL enhancing the signal of 2.48× in comparison with the monomeric form, Ib-1-biot at a dilution of 1/1000 (FIG. 1E).

Surrounding Part of Epitope-Determining Sequence for aPL is Essential for the Proper Interactions with Epitope

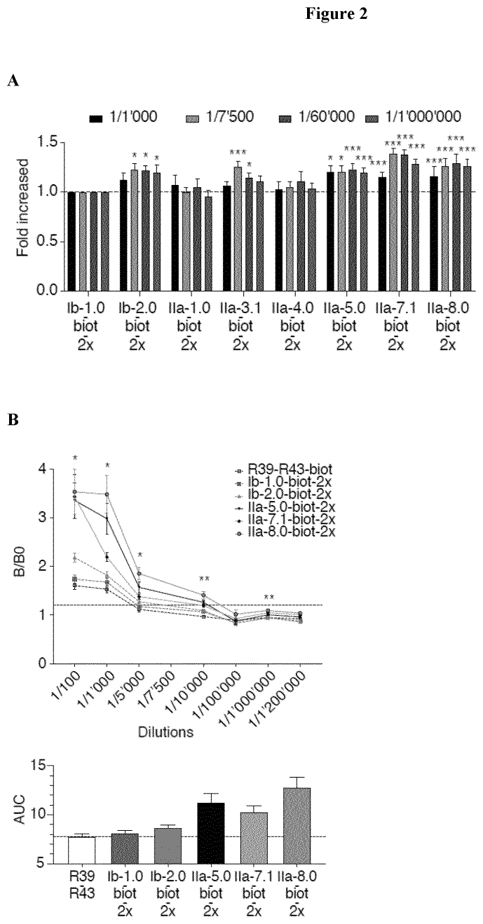

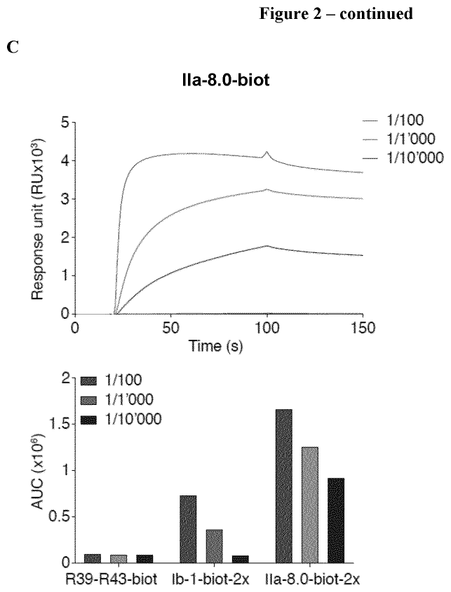

It was performed a peptide substitution scan of Ib-1-biot VSRGGMRKKKKPLTG (SEQ ID NO: 6) carrying the optimized epitope Ib-1-biot and based on an exchange of the underlined amino acid positions with the 20 main amino acids. The resulting peptide microarrays contained 140 different peptides. It was observed that the peptide substitution of the position n° 1, 2, 12 and 15 have significant influences on the ability to interact with aPL from patients 1, 2, 3, 4 and, in particular, form the plasma pool. Indeed, the substitution of the position 1, 12 or 15 by an Arginine (R) or a Lysine (K) increases the avidity of the peptide for all aPL from patients. However, from the amount of identified peptides showing the higher enhancement of aPL interactions, it seems that no individual mutations displayed a substantial improvement more than any another. In this context, the enhancement relative to Ib-1.0-biot-2x of identified peptides was evaluated (FIG. 2A). The peptides Ib-1.0-biot-2x, Ib-2.0-biot-2x, IIa-5.0-biot-2x, IIa-7.1-biot-2x and IIa-8.0-biot-2x have the ability to interact with aPL which is significantly increased with at least three of the four dilutions. Although the sequence IIa-3.1-biot-2x presents a significant increase at a dilution of 1/7,500 and 1/60,000, the increase is not enough to be selected as it can also be appreciated with the peptide IIa-1.0-biot-2x and IIa-4.0-biot-2x (FIG. 2A). However, streptavidin being known to produce unspecific binding, it was used the ratio between aPL and a pool of human plasma as control (B/B0) (FIG. 2B, upper panel). This representation therefore allows to distinguish the real levels of interactions. It was then performed a dilution assay from dilution of 1/100 to 1/1,200,000 and it was observed relevant differences between the different identified peptides. By comparison of area under the curve (AUC), it was possible to detect the most prone-interacting peptide with aPL. Thus, the peptide IIa-8.0-biot-2x obtained the higher AUC (FIG. 2B, bottom panel). To formally quantify the ability of aPL to interact with IIa-8.0-biot-2x, it was monitored by flowing various concentrations of aPL over a IIa-8.0-biot-2x-coated chip surface (FIG. 2C). These SPR experiments performed with IIa-8.0-biot-2x-coated chip surface show that the avidity of aPL is 1.3×more at all dilutions than its avidity for Ib-1.0-biot-2x (FIG. 2C, upper panel, and FIG. 1D). The sustained stability of binding is also significantly increased as it could be observed through the shape of the curves (FIG. 2C, upper panel). In FIG. 2C, bottom panel, it can be appreciated more precisely the improvement of the quality of interactions. Indeed, AUC is 2.3×, 3.43× and 10.5× higher with IIa-8.0-biot-2x than with Ib-1.0-biot-2x at dilutions of 1/100, 1/1,000 and 1/10,000, respectively (FIG. 2C, bottom panel). Lastly, it was observed that AUC for peptide IIa-8.0-biot-2x is more of 10× the value obtained with the initial target, i.e. R39-43. Altogether these results demonstrate that aPL from Human patients though recognizing R39-R43 epitope on 132GP1, has a stronger affinity for an epitope rich in Arginine (R) and Lysine (K) which are present in crucial locations along the sequence.

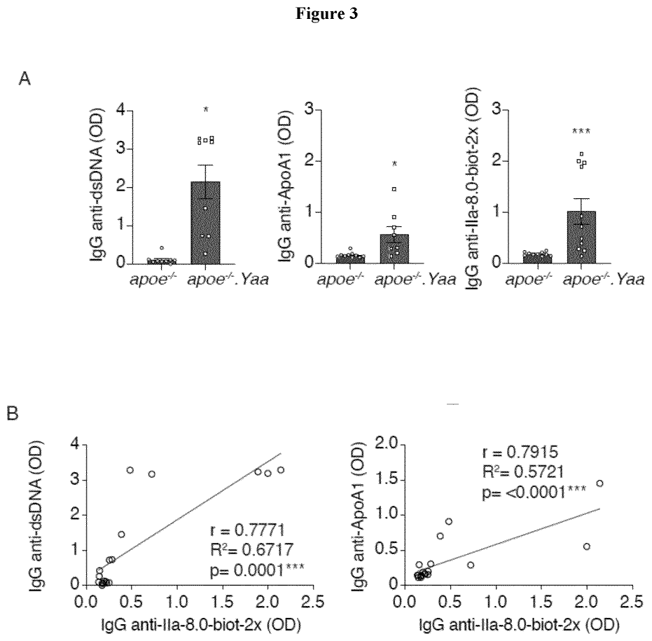

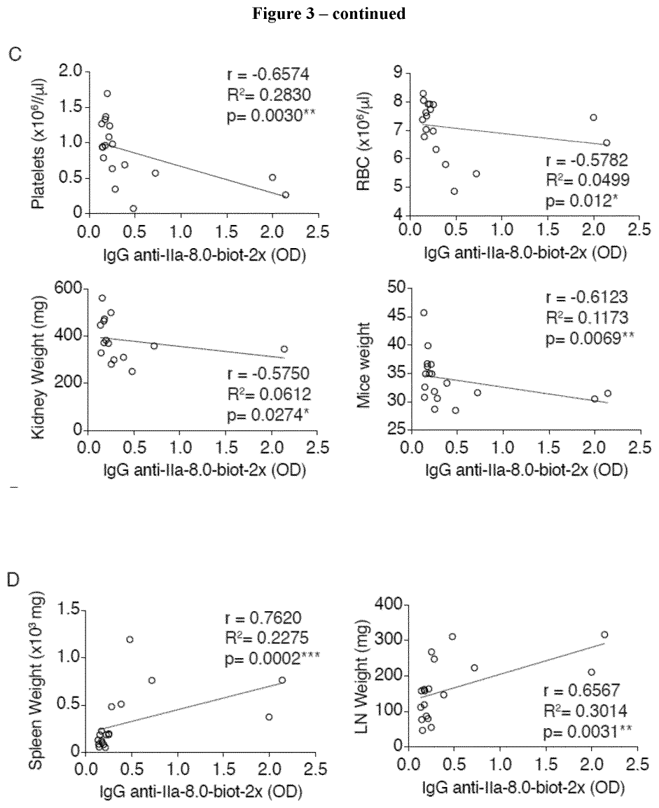

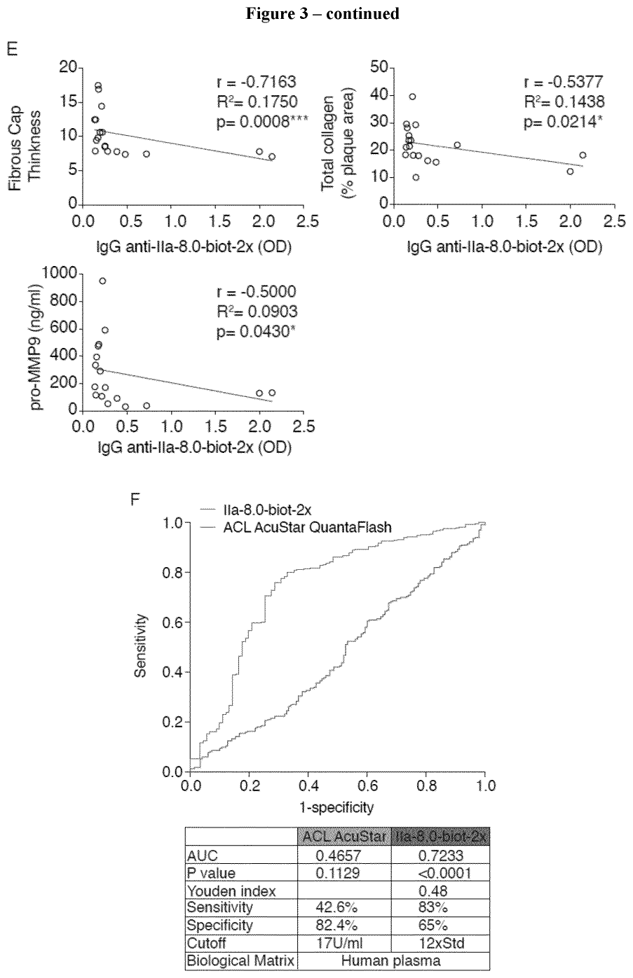

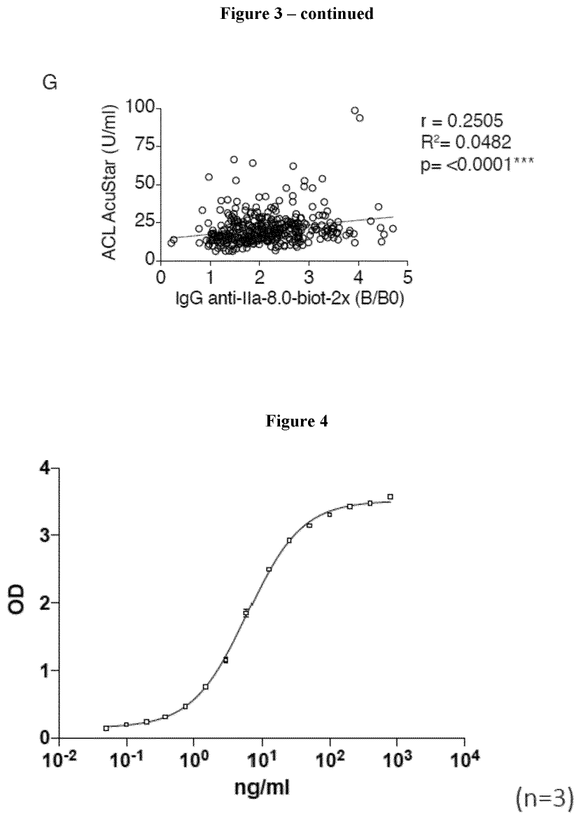

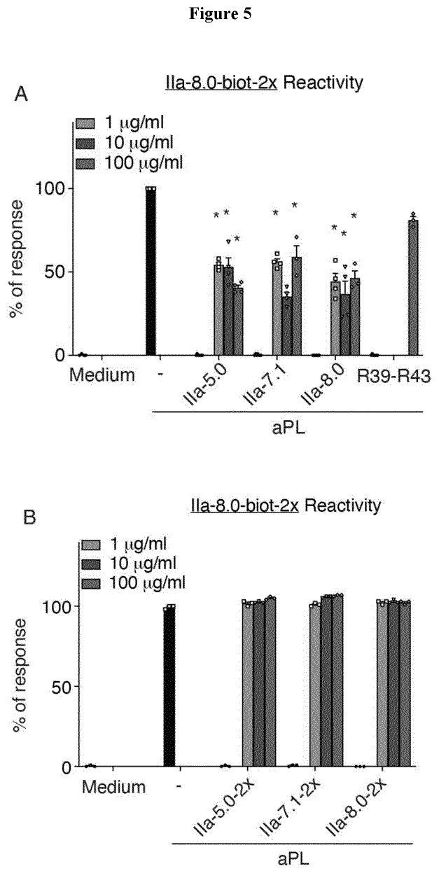

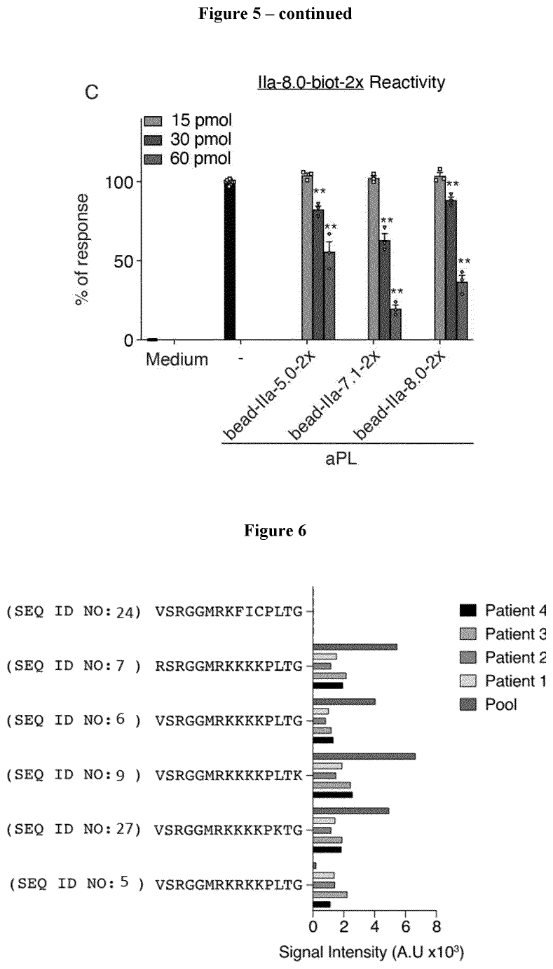

Levels of circulating IgG anti-8.0-biot-2x in lupus-prone mouse model and Human cohort are strongly correlated with clinical manifestation of APS— Inventors recently studied the mechanism leading to higher cardiovascular (CV) mortality in systemic lupus erythematous (SLE). In this context, the association between autoantibodies, atherosclerotic parameters and plaque vulnerability was investigated. To address this issue, it was crossed the lupus-prone Nba2.Yaa mouse model with atherosclerosis-prone apoE^{-/-} mice, thus generating a mouse model (apoe^{-/-}.Nba2.Yaa) that enabled the study in vivo of the potential relation between autoantibodies and atherosclerotic plaque vulnerability. APS occurs alone or in association with other autoimmune diseases, particularly systemic lupus erythematosus (SLE), i.e. 50% SLE patients have APS. It was thus investigated whether lupus-prone mouse model carried IgG anti-IIa-8.0-biot-2x in correlation with other IgG autoantibodies as well as clinical manifestations of APS and atherosclerotic plaque vulnerability. The levels of IgG autoantibodies against dsDNA, ApoA1 and IIa-8.0-biot-2x were measured (FIG. 3A). As mentioned in previous article, the levels of IgG anti-dsDNA and IgG anti-ApoA1 are increased in apoe^{-/-}.Nba2.Yaa in comparison with apoe^{-/-} mice. Beyond the fact that IgG anti-IIa-8.0-biot-2x is also significantly produced by apoe^{-/-}.Nba2.Yaa, identified peptide IIa-8.0-biot-2x is able to interact also with aPL produced by mice (FIG. 3A). Interestingly, the productions of IgG anti-dsDNA and anti-ApoA1 are strongly correlated with IgG anti-IIa-8.0-biot-2x (FIG. 3B). Other typical clinical manifestations of APS are inversely correlated with the presence of IgG anti-IIa-8.0-biot-2x. Obtained data indicate that the counts of platelets and red blood cells (RBC) are low when the concentration of IgG anti-IIa-8.0-biot-2x is high (FIG. 3C). These effects could be also noticed concerning kidney and mice weight (FIG. 3D) Interestingly, a correlation between IgG anti-IIa-8.0-biot-2x concentration and the weight of the spleen and lymph nodes is also observed (FIG. 3D). In regard to the atherosclerotic plaque vulnerability parameters, fibrous cap thickness, total collagen and circulating pro-MMP9 are inversely correlated with the level of IgG anti-IIa-8.0-biot-2x (FIG. 3E). Finally, evaluation of diagnostic tests for APS is a particular matter of concern, not only for confirming the presence of disease but also to rule out the disease in healthy subjects. Consequently, it was performed a receiver operating characteristic (ROC) curve analysis for rating inventors' custom ELISA results versus the gold standard, i.e. ACL AcuStar QuantaFlash. It could be seen that ROC curve obtained with inventors' custom ELISA on a cohort of 380 Human patients shows a greater discriminant capacity than ACL AcuStar QuantaFlash (FIG. 3F, upper panel). Although the specificity of inventors' custom ELISA is slightly inferior of AcuStar QuantaFlash, the sensitivity has almost doubled and the correlation between both assay is well significative (FIG. 3F, bottom panel, and FIG. 3G). Obtained data indicate that IgG anti-IIa-8.0-biot-2x is highly relevant as APS biomarker through a good correlation with all clinical manifestations and IgG anti-β2GP1. They further show that IgG anti-IIa-8.0-biot-2x is potentially relevant for CV risk as similarly to anti-ApoA-1 IgG associated with a higher prevalence and incidence of coronary artery disease (CAD). Finally, although further developments are required to improve sensitivity and specificity of immunoassay, new diagnostic tools based on identified peptide IIa-8.0-biot-2x could lead to define the real negativity and improve the risk stratification of APS.

Monoclonal Antibody

Monoclonal antibody has been generated against the peptide construct of formula I and used as calibrator (calibrating antibody). The calibration curve from 50 pg/ml to 800 ng/ml shown in FIG. 4 can be observed.

Monoclonal IgG Antibody Generated with Anti-8.0-Biot-2x for the Standardization of ELISA

In order to develop a fully standardized ELISA and to confirm anti-IIa-8.0-biot-2x as the epitope of aPL conferring their proinflammatory properties, a monoclonal IgG antibody was generated in mice (14B10). To formally quantify the ability of 14B10 to interact with IIa-8.0-biot-2x, flowing of various concentrations of 14B10 over a IIa-8.0-biot-2x coated chip surface was monitored. These SPR experiments performed with IIa-8.0-biot-2x coated chip surface show that the constant of dissociation (Kd) of 14B10 is 180 pM, classifying this monoclonal IgG antibody amongst the very high affinity antibodies for IIa-8.0-biot-2x. The antibody 14B10 is also able to bind to the epitope R39-R43 with the similar affinity as the aPL It can be extrapolated from the sensorgram that aPL from APS patients has a constant of dissociation close (Kd) to 8.9 μM classifying aPL antibody amongst low to medium affinity antibodies for R39-R43. The best standard curve with 14B10 antibody was determined. The dynamic range of 800 ng/ml to 50 pg/ml is high and the cutoff value is present in the linear part of the curve (not shown). Altogether these data demonstrate that antibody 14B10 IgG has a high affinity and avidity for IIa-8.0-biot-2x but, although monoclonal, is also able to specifically bind the epitope R39-R43 with low affinity. Lastly, the standard curve performed with 14B10 shows a good dynamic range leading to fully standardized indirect immunoassay for the detection of anti-β2GP1 IgG.

Dimers and Monomers of IIa-5.0, IIa-7.1 and IIa-8.0 Peptides are Able to Inhibit the Binding Activity of aPL In Vitro

To examine the functional ability of IIa-5.0, IIa-7.1, IIa-8.0 peptides and R39-R43 to inhibit the activity of pool of aPL isolated from APS patients, the remaining reactivity of aPL was evaluated for IIa-8.0-biot-2x which had previously been treated with increasing concentration of peptides. While the treatment of aPL with monomeric peptides IIa-5.0, IIa-7.1 and IIa-8.0 prevents their further binding to IIa-8.0-biot-2x ELISA (FIG. 7A), the incubation of aPL with dimer of IIa-5.0-2x, IIa-7.1-2x and IIa-8.0-2x have no effect on aPL ability to interact with Ha-8.0-biot-2x ELISA (FIG. 7B). Of note that the monomeric peptide R39-R43 failed to significantly inhibit the interaction of aPL with IIa-8.0-biot-2x ELISA (FIG. 7A). The flag-type orientation prevents potential interactions with the plate or with itself, particularly in solution. Streptavidin magnetic beads associated with IIa-5.0-biot-2x, IIa-7.1-biot-2x and Ha-8.0-biot-2x peptides have been generated. aPL was treated with increasing concentrations of beads before to measure the remaining binding activity of aPL on IIa-8.0-biot-2x ELISA (FIG. 7C). We can observe that the ability of aPL to interact with IIa-8.0-biot-2x is dose dependently inhibited by peptides associated-beads (FIG. 7C). These results further confirm the importance of the orientation for efficient interactions with pathogenic aPL.

Inhibition Experiments

To assess the ability of IIa-5.0-2x, IIa-7.1-2x and IIa-8.0-2x to inhibit in vitro the binding of aPL to IIa-8.0-biot-2x. aPL pool sera were preincubated for 90 min at RT with increasing concentrations (1, 10 and 10011 g/ml) of monomeric, dimeric IIa-5.0, IIa-7.1 and IIa-8.0 peptides and of IIa-5.0-biot-2x, IIa-7.1-biot-2x and IIa-8.0-biot-2x-associated beads (15, 30 and 60 pmol). 120 pmol correspond to the amount of IIa-8.0-biot-2x coated in well of Streptavidin Coated High Capacity Plates 96 well plates (Thermofisher). After the pre-incubation time, the serum was added anti-b2GP1 IgG (anti-IIa-8.0-2x) ELISA according to the protocol.

Discussion/Conclusion

Inventors have established that the residues between K44 and P48 are critical to increase the avidity for aPL from APS patients. The substitution of these three residues by a Lysine or

Arginine enhanced the avidity of aPL by almost 6 times. Inventors also demonstrated that the amino-acids surrounding the epitope R39-K47 have a function for the interaction with aPL. The modification of the sequences -VSR- and -PLTG- could thus influence positively or negatively the binding of aPL (data not shown).

While aPL has a higher affinity for peptide IIa-8.0-biot-2x in comparison to the wildtype Domain I and in regards of the data obtained by inventors, peptide IIa-8.0-biot-2x, IIa-5.0-biot-2x and IIa-7.1-biot-2x could be used for specific clinical management of APS.

The results in present disclosure demonstrate that sequence with the highest aPL-binding activity possess a length of 15 residues with Lysin rich region.

1. An isolated peptide comprising the amino acid sequence X₁SRGGMRKX₂KKX₃X₄TX₅ (SEQ ID NO: 1), wherein
X₁ is R or V,
X₂ is R or K,
X₃ is P or K,
X₄ is L or K,
X₅ is G or K.

2. The isolated peptide of claim 2, wherein the isolated peptide
(SEQ ID NO: 2)
RSRGGMRKRKKPLTG,
(SEQ ID NO: 3)
VSRGGMRKRKKPLTK,
(SEQ ID NO: 4)
VSRGGMRKRKKKKTG,
(SEQ ID NO: 5)
VSRGGMRKRKKPLTG,
(SEQ ID NO: 6)
VSRGGMRKKKKPLTG,
(SEQ ID NO: 7)
RSRGGMRKKKKPLTG,
(SEQ ID NO: 8)
VSRGGMRKKKKKKTG,
or
(SEQ ID NO: 9)
VSRGGMRKKKKPLTK.

3. A peptide construct of formula I
S₂—P—S₁—P (I)
wherein
P is a peptide consisting of the amino acid sequence X₁SRGGMRKX₂KKX₃X₄TX₅ (SEQ ID NO: 1), wherein
X₁ is R or V,
X₂ is R or K,
X₃ is P or K,
X₄ is L or K,
X₅ is G or K,
S₂ is absent or is a spacer peptide sequence or a polymer, wherein the spacer peptide sequence is a poly-Gly spacer consisting of 3-16 glycines or a 3 to 16-amino acids Gly-rich spacer; and
S₁ is a spacer peptide sequence or a polymer, wherein the spacer peptide sequence is a poly-Gly spacer consisting of 3-16 glycines or a 3 to 16-amino acids Gly-rich spacer.

4. The peptide construct of claim 3, wherein P for each occurrence is independently
(SEQ ID NO: 2)
RSRGGMRKRKKPLTG,
(SEQ ID NO: 3)
VSRGGMRKRKKPLTK,
(SEQ ID NO: 4)
VSRGGMRKRKKKKTG,
(SEQ ID NO: 5)
VSRGGMRKRKKPLTG,
(SEQ ID NO: 6)
VSRGGMRKKKKPLTG,
(SEQ ID NO: 7)
RSRGGMRKKKKPLTG,
(SEQ ID NO: 8)
VSRGGMRKKKKKKTG,
or
(SEQ ID NO: 9)
VSRGGMRKKKKPLTK.

5. (canceled)

6. A method for detecting the presence of antiphospholipid antibody in a sample comprising:
(i) contacting the sample with the peptide of claim 1 or a peptide construct comprising the peptide immobilized on a surface or on under conditions allowing for the formation of a complex between antiphospholipid antibodies with the peptide or the peptide construct; and
(ii) detecting the complex using an immunoassay.

7. The method of claim 6, wherein the immunoassay is an ELISA, a Lateral Flow Assay (LFA), an immunoprecipitation, an enzyme immunoassay (EIA), a radioimmunoassay (RIA), a fluorescence immunoassay, a chemiluminescent assay, an agglutination assay, a nephelometric assay, a turbidimetric assay, a Western blot, a competitive immunoassay, a non-competitive immunoassay, a homogeneous immunoassay, a heterogeneous immunoassay, a bioassay, or a reporter-assay such as a Luciferase-Assay.

8. An antibody or an antigen-binding fragment thereof, that binds to the peptide of claim 1 or a peptide construct comprising the peptide, wherein the antibody or the antigen binding fragment thereof comprises a heavy chain variable region that comprises CDR1, CDR2, and CDR3 domains; and a light chain variable region that comprises CDR1, CDR2, and CDR3 domains, wherein the heavy chain variable region CDR1, CDR2, and CDR3 sequences are as set forth in SEQ ID NO: 16, SEQ ID NO: 17, and SEQ ID NO: 18, respectively; and the light chain variable region CDR1, CDR2, and CDR3 sequence are as set forth in SEQ ID NO: 19, SEQ ID NO: 20, and SEQ NO: 21, respectively.

9. (canceled)

10. A kit for detecting in a sample the presence or absence of an antiphospholipid antibody, the kit comprising the peptide of claim 1 or a peptide construct comprising the peptide.

11. A device for detecting in a sample the presence or absence of an antiphospholipid antibody, the device comprising a solid support comprising the peptide of claim 1 or a peptide construct comprising the peptide.

12. A pharmaceutical composition comprising the peptide of claim 1 or a peptide construct comprising the peptide in an amount effective to reduce or inhibit one or more symptoms of antiphospholipid syndrome (APS), and a pharmaceutically acceptable carrier.

13. A method for reducing or inhibiting one or more symptoms of the antiphospholipid syndrome (APS) in a subject in need thereof, the method comprising administering the pharmaceutical composition of claim 12 to the subject.

14. A method of selectively removing antiphospholipid antibodies from blood, serum or plasma, the method comprising:
immobilizing the peptide of claim 1 or a peptide construct comprising the peptide to an immunoaffinity membrane, and
passing the blood, serum or plasma through the immunoaffinity membrane so that antiphospholipid antibodies from the blood, serum or plasma will be removed by the immunoaffinity membrane.

15. The method of claim 6, wherein the peptide construct is a peptide construct of formula I
S₂—P—S₁—P (I)
wherein
P is a peptide consisting of the amino acid sequence X₁SRGGMRKX₂KKX₃X₄TX₅ (SEQ ID NO: 1), wherein
X₁ is R or V,
X₂ is R or K,
X₃ is P or K,
X₄ is L or K,
X₅ is G or K;
S₂ is absent or is a spacer peptide sequence or a polymer, wherein the spacer peptide sequence is a poly-Gly spacer consisting of 3-16 glycines or a 3 to 16-amino acids Gly-rich spacer; and
S₁ is a spacer peptide sequence or a polymer, wherein the spacer peptide sequence is a poly-Gly spacer consisting of 3-16 glycines or a 3 to 16-amino acids Gly-rich spacer.

16. The antibody or an antigen-binding fragment thereof of claim 8, wherein the antibody or an antigen-binding fragment thereof binds the peptide construct comprising the peptide and wherein the peptide construct is a peptide construct of formula I
S₂—P—S₁—P (I)
wherein
P is a peptide consisting of the amino acid sequence X₁SRGGMRKX₂KKX₃X₄TX₅ (SEQ ID NO: 1), wherein
X₁ is R or V,
X₂ is R or K,
X₃ is P or K,
X₄ is L or K,
X₅ is G or K;
S₂ is absent or is a spacer peptide sequence or a polymer, wherein the spacer peptide sequence is a poly-Gly spacer consisting of 3-16 glycines or a 3 to 16-amino acids Gly-rich spacer; and
S₁ is a spacer peptide sequence or a polymer, wherein the spacer peptide sequence is a poly-Gly spacer consisting of 3-16 glycines or a 3 to 16-amino acids Gly-rich spacer.

17. The kit of claim 10, wherein the peptide construct is a peptide construct of formula I
S₂—P—S₁—P (I)
wherein
P is a peptide consisting of the amino acid sequence X₁SRGGMRKX₂KKX₃X₄TX₅ (SEQ ID NO: 1), wherein
X₁ is R or V,
X₂ is R or K,
X₃ is P or K,
X₄ is L or K,
X₅ is G or K;
S₂ is absent or is a spacer peptide sequence or a polymer, wherein the spacer peptide sequence is a poly-Gly spacer consisting of 3-16 glycines or a 3 to 16-amino acids Gly-rich spacer; and
S₁ is a spacer peptide sequence or a polymer, wherein the spacer peptide sequence is a poly-Gly spacer consisting of 3-16 glycines or a 3 to 16-amino acids Gly-rich spacer.

18. The device of claim 11, wherein the peptide construct is a peptide construct of formula I
S₂—P—S₁—P (I)
wherein
P is a peptide consisting of the amino acid sequence X₁SRGGMRKX₂KKX₃X₄TX₅ (SEQ ID NO: 1), wherein
X₁ is R or V,
X₂ is R or K,
X₃ is P or K,
X₄ is L or K,
X₅ is G or K;
S₂ is absent or is a spacer peptide sequence or a polymer, wherein the spacer peptide sequence is a poly-Gly spacer consisting of 3-16 glycines or a 3 to 16-amino acids Gly-rich spacer; and
S₁ is a spacer peptide sequence or a polymer, wherein the spacer peptide sequence is a poly-Gly spacer consisting of 3-16 glycines or a 3 to 16-amino acids Gly-rich spacer.

19. The pharmaceutical composition of claim 12, wherein the peptide construct is a peptide construct of formula I
S₂—P—S₁—P (I)
wherein
P is a peptide consisting of the amino acid sequence X₁SRGGMRKX₂KKX₃X₄TX₅ (SEQ ID NO: 1), wherein
X₁ is R or V,
X₂ is R or K,
X₃ is P or K,
X₄ is L or K,
X₅ is G or K;
S₂ is absent or is a spacer peptide sequence or a polymer, wherein the spacer peptide sequence is a poly-Gly spacer consisting of 3-16 glycines or a 3 to 16-amino acids Gly-rich spacer; and
S₁ is a spacer peptide sequence or a polymer, wherein the spacer peptide sequence is a poly-Gly spacer consisting of 3-16 glycines or a 3 to 16-amino acids Gly-rich spacer.

20. The method of claim 14, wherein the peptide construct is a peptide construct of formula I
S₂—P—S₁—P (I)
wherein
P is a peptide consisting of the amino acid sequence X₁SRGGMRKX₂KKX₃X₄TX₅ (SEQ ID NO: 1), wherein
X₁ is R or V,
X₂ is R or K,
X₃ is P or K,
X₄ is L or K,
X₅ is G or K;
S₂ is absent or is a spacer peptide sequence or a polymer, wherein the spacer peptide sequence is a poly-Gly spacer consisting of 3-16 glycines or a 3 to 16-amino acids Gly-rich spacer; and
S₁ is a spacer peptide sequence or a polymer, wherein the spacer peptide sequence is a poly-Gly spacer consisting of 3-16 glycines or a 3 to 16-amino acids Gly-rich spacer.


SEQUENCE LISTING (27 sequences)

SEQ ID NO: 1 - protein, 15 aa, Artificial Sequence
  Synthetic sequence
  MISC_FEATURE (1)..(1): X1 is R or V
  MISC_FEATURE (9)..(9): X2 is R or K
  MISC_FEATURE (12)..(12): X3 is P or K
  MISC_FEATURE (13)..(13): X4 is L or K
  MISC_FEATURE (15)..(15): X5 is G or K
XSRGGMRKXKKXXTX

SEQ ID NO: 2 - protein, 15 aa, Artificial Sequence
  Synthetic sequence
RSRGGMRKRKKPLTG

SEQ ID NO: 3 - protein, 15 aa, Artificial Sequence
  Synthetic sequence
VSRGGMRKRKKPLTK

SEQ ID NO: 4 - protein, 15 aa, Artificial Sequence
  Synthetic sequence
VSRGGMRKRKKKKTG

SEQ ID NO: 5 - protein, 15 aa, Artificial Sequence
  Synthetic sequence
VSRGGMRKRKKPLTG

SEQ ID NO: 6 - protein, 15 aa, Artificial Sequence
  Synthetic sequence
VSRGGMRKKKKPLTG

SEQ ID NO: 7 - protein, 15 aa, Artificial Sequence
  Synthetic sequence
RSRGGMRKKKKPLTG

SEQ ID NO: 8 - protein, 15 aa, Artificial Sequence
  Synthetic sequence
VSRGGMRKKKKKKTG

SEQ ID NO: 9 - protein, 15 aa, Artificial Sequence
  Synthetic sequence
VSRGGMRKKKKPLTK

SEQ ID NO: 10 - protein, 16 aa, Artificial Sequence
  Synthetic sequence
GGGGSLVPRGSGGGGS

SEQ ID NO: 11 - protein, 6 aa, Artificial Sequence
  Synthetic sequence
GSGSGS

SEQ ID NO: 12 - protein, 11 aa, Artificial Sequence
  Synthetic sequence
GSGGGTGGGSG

SEQ ID NO: 13 - protein, 10 aa, Artificial Sequence
  Synthetic sequence
GGGGSGGGGS

SEQ ID NO: 14 - protein, 5 aa, Artificial Sequence
  Synthetic sequence
GGGGS

SEQ ID NO: 15 - protein, 10 aa, Artificial Sequence
  Synthetic sequence
GSGSGTGSGS

SEQ ID NO: 16 - protein, 5 aa, Artificial Sequence
  Synthetic sequence
SYWIQ

SEQ ID NO: 17 - protein, 17 aa, Artificial Sequence
  Synthetic sequence
AIYPGDGDTSYTQKFKG

SEQ ID NO: 18 - protein, 12 aa, Artificial Sequence
  Synthetic sequence
LGDGYDDYAMDY

SEQ ID NO: 19 - protein, 15 aa, Artificial Sequence
  Synthetic sequence
RASESVDSYGNSFMH

SEQ ID NO: 20 - protein, 7 aa, Artificial Sequence
  Synthetic sequence
LASNLES

SEQ ID NO: 21 - protein, 9 aa, Artificial Sequence
  Synthetic sequence
QQNNEDPYT

SEQ ID NO: 22 - protein, 121 aa, Artificial Sequence
  Synthetic sequence
QVQLQQSGAELARPGASVKLSCKASGYTFSSYWIQWVKQRPGQGLEWIGAIYPGDGDTSYTQKFKGKATLTADKSSSTAYMQLSSLASEDSAVYYCARLGDGYDDYAMDYWGQGTSVTVSS

SEQ ID NO: 23 - protein, 111 aa, Artificial Sequence
  Synthetic sequence
NIVLTQSPASLAVSLGQRATISCRASESVDSYGNSFMHWYQQKPGQPPKLLIYLASNLESGVPARFSGSGSRTDFTLTIDPVEADDVATYYCQQNNEDPYTFGGGTKLEIK

SEQ ID NO: 24 - protein, 15 aa, Artificial Sequence
  Synthetic sequence
VSRGGMRKFICPLTG

SEQ ID NO: 25 - protein, 15 aa, Artificial Sequence
  Synthetic sequence
VSRGGMRKFIKPLTG

SEQ ID NO: 26 - protein, 15 aa, Artificial Sequence
  Synthetic sequence
  MISC_FEATURE (1)..(1): X5 is G or K
  MISC_FEATURE (3)..(3): X4 is L or K
  MISC_FEATURE (4)..(4): X3 is P or K
  MISC_FEATURE (7)..(7): X2 is R or K
  MISC_FEATURE (15)..(15): X1 is R or V
XTXXKKXKRMGGRSX

SEQ ID NO: 27 - protein, 15 aa, Artificial Sequence
  Synthetic sequence
VSRGGMRKKKKPKTG